(19) United States
(12) Patent Application Publication
(10) Pub. No.: US 2021/0155971 A1
(43) Pub. Date: May 27, 2021

(54) METHODS OF DETECTING AMINO ACID DEFICIENCIES

(71) Applicant: PACIFIC NORTHWEST RESEARCH INSTITUTE, Seattle, WA (US)

(72) Inventors: Amy Sirr, Walla Walla, WA (US); Adrian C. Scott, Deming, WA (US); Aimee M. Dudley, Seattle, WA (US)

(21) Appl. No.: 17/047,667

(22) PCT Filed: Apr. 16, 2019
(86) PCT No.: PCT/US2019/027658

§ 371 Date: Oct. 14, 2020

Related U.S. Application Data
(60) Provisional application No. 62/659,307, filed on Apr. 18, 2018.

(51) Int. Cl. C12Q 1/52; C12N 15/81; C12Q 1/02; C12Q 1/6883

(52) U.S. Cl. CPC C12Q 1/52; C12N 15/81; C12Q 1/02; G01N 2800/52; C12Q 1/6883; G01N 2800/04; C12Q 2600/156; C12Y 206/01052

(57) ABSTRACT

The present application relates to a method of screening a subject (and/or treating a subject) for a disease. The application further relates to cells and kits for determination of a disease. Also contemplated are treatments, including those based on a personalized cell model system that determines a subject's threshold for a disease and their personalized treatment.

### INCORPORATION BY REFERENCE TO ANY PRIORITY APPLICATIONS

This applications claims the benefit of priority to U.S. provisional Patent Application No. 62/659,307, filed Apr. 18, 2018 and titled “METHODS OF DETECTING AMINO ACID DEFICIENCIES”, which is incorporated by reference herein in its entirety for all purposes

### SEQUENCE LISTING AND TABLES IN ELECTRONIC FORMAT

The present application is being filed along with a Sequence Listing in electronic format. The Sequence Listing is provided as a file entitled SequenceListingPNDRI008, created Apr. 16, 2019 which is 13,175 bytes in size. The information in the electronic format of the Sequence Listing is incorporated herein by reference in its entirety.

### FIELD

Methods of determining amino acid deficiencies in a subject, such as a developing fetus or an asymptomatic subject. Also contemplated are methods for predicting genetic outcome of progeny that is directed to probability of metabolic diseases. Method of therapy and kits are also provided.

### BACKGROUND

Individuals with homozygous or compound heterozygous loss of function mutations in any of three metabolic genes responsible for the biosynthesis of serine may have a severe neurodevelopmental syndrome that presents with congenital microcephaly, intractable seizures, and severe psychomotor retardation.

Neu-Laxova syndrome (NLS) is a rare genetic disorder that is inherited as an autosomal recessive trait. The syndrome is characterized by severe growth delays that occur before birth. Intrauterine growth retardation, low birth weight and length, and distinctive abnormalities of the head and facial (craniofacial) region are some of the symptoms of this disorder. Other characteristics may include a marked smallness of the head (microcephaly), sloping of the forehead, widely spaced eyes (ocular hypertelorism), and other malformations, resulting in a distinctive facial appearance. Abnormal accumulations of fluid in tissues throughout the body (generalized edema), permanent flexion and immobilization of multiple joints (flexion contractures), limb malformations and/or abnormalities of the brain, skin, genitals, kidneys, and/or heart may also be found in those suffering from Neu-Laxova syndrome (NLS). (Scott et. al; Am J Med Genet. 1981; 9:165-75; included by reference)

In newborns additional physical abnormalities may also be present, such as underdevelopment of the genitals, the absence of one of the kidneys (unilateral renal agenesis) or other renal defects, underdevelopment of the lungs (pulmonary hypoplasia), or structural abnormalities of the heart (congenital heart defects). Congenital cardiac malformations may include an abnormal opening in the fibrous partition (septum) that separates the upper or lower chambers of the heart (atrial or ventricular septal defects); persistence of the fetal opening between the two major arteries (aorta, pulmonary artery) emerging from the heart (patent ductus arteriosus), and/or a heart defect in which the aortic and pulmonary arteries are in one another's normal positions. Infants with NLS may be stillborn or develop life-threatening complications shortly after birth.

NLS is transmitted as an autosomal recessive trait. Thus, the risk of transmitting the disease to the children of a couple, both of whom are carriers for a recessive disorder, is 25 percent. Fifty percent of their children risk being carriers of the disease, but generally will not show symptoms of the disorder. Twenty-five percent of their children may receive both normal genes, one from each parent, and will be genetically normal (for that particular trait).

### SUMMARY OF THE INVENTION

When metabolic diseases are diagnosed early, dietary supplementation can reduce or even eliminate disease symptoms. Prenatal diagnosis followed by serine supplementation to the mother starting early in pregnancy and to the patient from birth onward may prevent or ameliorate the onset of symptoms.

By determining which alleles of the serine biosynthetic genes are neutral or pathogenic, the assays described herein would facilitate a gene-sequencing based diagnostic that can be used earlier (including on the developing fetus or the asymptomatic parents). This can be used to determine the genetic outcome of a fetus, or the information may be used to determine the genetic outcome for future progeny of two subjects. The alleles may also be used to develop a personalized therapeutic therapy or to guide intervention to ameliorate symptoms of disease, e.g. amino acid supplementation that may reduce or prevent morbidity and/or mortality associated with the disease.

The biochemical pathway that produces serine is nearly identical between humans and yeast. Loss of function mutations in any of the three serine biosynthetic genes in humans cause Serine Deficiency Syndrome. In yeast, loss of function of the yeast enzymes produces a quantitative and easily measured trait, poor growth in the absence of serine supplementation. In the embodiments herein, it is shown that the human protein coding sequence of the PSAT1 gene can functionally replace the corresponding SER1 gene in yeast. Thus the yeast-based assay described in the embodiments for measuring the activity of PSAT1 gene variants found in a specific patient may be used for a genome-wide association study (GWAS), or the population at large.

In a first aspect, a method of screening a subject for a disease is provided. The method comprises the steps: obtaining genomic DNA from the subject, identifying a gene of interest from the genomic DNA, inserting the gene into a construct, wherein the construct is a linear DNA, providing a test cell, wherein the test cell has a homologous gene of the gene of interest, wherein the homologous gene has been knocked out of the test cell, introducing the construct into the test cell, evaluating the test cell for cell growth in a media, wherein the media lacks an amino acid, wherein the test cell growth is compared to a control cell that has the homologous gene, wherein when test cell growth is comparable to that of the control cell it indicates that the gene of interest encodes a functional protein, and wherein when the test cell growth is slow as compared to the control cell it indicates that the gene of interest encodes a non-functional protein or protein with decreased function and indicates the disease. In some embodiments, the subject is a fetus, neonate, juvenile or adult. In some embodiments, the cell and the control cell are yeast cells. In some embodiments, the amino acid is serine. In some embodiments, the subject is pregnant. In some embodiments, the gene encodes 3-PGDH, PSAT1 or PSPH. In some embodiments, cell growth is measured by optical density of a liquid culture, or a number of pixels of a colony of cells growing on solid media. In some embodiments, wherein the evaluating step comprises measuring cell growth for 0.5, 2, 4, 6, 8, 10, 12, 24, 36, 72, 96 or 120 hours or any number of hours in between a range defined by any two aforementioned values. In some embodiments, growth of the test cell is comparable to that of the control cell by automated image analysis, wherein the test cell has a growth value that is at least 90% of the growth value of the control cell. In some embodiments, cell growth of the test cell is slow as compared to the control cell, wherein the cell has an optical density that is 79% or less than the growth value of the control cell. In some embodiments, the gene of interest is further analyzed for a single-nucleotide polymorphism. In some embodiments, the single-nucleotide polymorphism is identified as being associated with loss of function or decreased function of a protein encoded by the gene of interest. In some embodiments, a t-test is performed between the test cell and control cell to examine significant growth difference, wherein a t-test p-values <0.0001 indicates a highly significant growth difference between the test cell and control cell. In some embodiments, the significant growth difference indicates that the gene of interest encodes a non-functional protein or protein with decreased function and indicates the disease. In some embodiments, the disease is NLS, Serine Deficiency Syndrome or retinal neuropathy. In some embodiments, the evaluating further comprises quantifying cell growth by photographing yeast growing on solid agar plates and performing image analysis using custom software.

In a second aspect, a method of determining amino acid deficiency in a subject is provided. The method comprises the steps: isolating genomic DNA from a subject, detecting a gene from the genomic DNA, wherein the gene encodes an enzyme of an amino acid synthesis pathway, inserting the gene into a construct, introducing the construct into a test cell, growing up the cell in a media absent of an amino acid and analyzing growth of the test cell in a culture, wherein the growth of the test cell is compared to a control cell, wherein the control cell is not deficient in amino acid synthesis wherein the test cell growth is compared to the control cell, wherein cell growth of the test cell is comparable to that of the control cell indicates that the gene encodes a functional enzyme and wherein cell growth of the test cell is slow as compared to the control cell indicates that the gene encodes a non-functional protein or protein with decreased function and indicates The method of claim 1, wherein the subject is a fetus, neonate, juvenile or adult. In some embodiments, the cell and the control cell are yeast cells. In some embodiments, the amino acid is serine. In some embodiments, the subject is pregnant. In some embodiments, the gene encodes 3-PGDH, PSAT1 or PSPH. In some embodiments, cell growth is measured by optical density. In some embodiments, the analyzing step comprises measuring cell growth for 0.5, 2, 4, 6, 8, 10, 12, 24, 36, 72, 96 or 120 hours or any number of hours in between a range defined by any two aforementioned values.

In some embodiments, cell growth of the test cell is comparable to that of the control cell by automated image analysis, wherein the test cell has a growth value that is at least 90% of the growth value of the control cell. In some embodiments, cell growth of the test cell is slow as compared to the control cell, wherein the cell has a growth value that is 79% or less than the optical density of the control cell.

In some embodiments, the gene of interest is further analyzed for a single-nucleotide polymorphism. In some embodiments, the single-nucleotide polymorphism is identified as being associated with loss of function or decreased function of a protein encoded by the gene of interest.

In some embodiments, a t-test is performed between the test cell and control cell to examine significant growth difference, wherein a t-test p-values <0.0001 indicates a highly significant growth difference between the test cell and control cell. In some embodiments, the significant growth difference indicates that the gene of interest encodes a non-functional protein or protein with decreased function and indicates the disease.

In some embodiments, the disease is NLS, Serine Deficiency Syndrome or retinal neuropathy or an amino acid deficiency in the subject.

In some embodiments, the analyzing step further comprises identifying at least one mutation in the gene.

In a third aspect, a method of determining a carrier of an amino acid deficiency disorder is provided. The method comprises the steps: isolating genomic DNA from a subject, detecting a gene from the genomic DNA, wherein the gene encodes an enzyme of an amino acid synthesis pathway, wherein the subject has two different alleles of the gene, inserting the gene into a construct, wherein the construct is a linear DNA, introducing the construct into a test cell, growing up the test cell in a media absent of an amino acid and evaluating the test cell for cell growth in a media, wherein the media lacks an amino acid, wherein test cell growth is compared to a control cell that has the homologous gene, wherein test cell growth of the cell is comparable to that of the control cell indicates that the gene of interest encodes a functional protein and wherein cell growth of the test cell is slow as compared to the control cell indicates that the gene of interest encodes a non-functional protein or protein with decreased function and indicates that the subject is a carrier of an amino acid deficiency disorder. In some embodiments, the amino acid deficiency disorder is NLS.

In a fourth aspect, a method of treating a subject with an amino acid deficiency is provided. The method comprises the steps: determining a subject or carrier of an amino acid deficiency disorder, wherein the determining comprises: detecting a gene from the genomic DNA, wherein the gene encodes an enzyme of an amino acid synthesis pathway; inserting the gene into a construct, wherein the construct is a linear DNA; introducing the construct into a test cell; growing up the test cell in a media absent of an amino acid; and evaluating the test cell for cell growth in a media, wherein the media lacks an amino acid, wherein test cell growth is compared to a control cell that has the homologous gene, wherein cell growth of the test cell is slow as compared to the control cell indicates that the gene of interest encodes a non-functional protein or protein with decreased function and indicates that the subject is a carrier of an amino acid deficiency disorder; and providing an adequate amount of an amino acid supplement to the subject. In some embodiments, the amino acid supplement is serine. In some embodiments, the enzyme is wherein the gene encodes 3-PGDH, PSAT1 or PSPH. In some embodiments, the subject is a fetus and wherein the mother of the fetus is provided an adequate amount of amino acid supplement. In some embodiments, the evaluating further comprises comparing growth of the test cell to a second control cell, wherein the second control cell is deficient in serine biosynthesis. In some embodiments, the amino acid deficiency disorder is NLS.

In a fifth aspect, a method of prenatal prediction of an amino acid deficiency is provided. The method comprises the steps: a) obtaining genomic DNA from a female and male subject, identifying a same gene of interest from the genomic DNA of the female and male subject, wherein the subjects are homozygous or heterozygous for the gene, inserting a first gene variant of interest from the female into a first construct, wherein the first construct is a linear DNA, inserting a second gene variant of interest from the male into a second construct, wherein the second construct is a linear DNA, providing a first cell, wherein the first cell has a homologous gene of the gene of interest, wherein the homologous gene has been knocked out of the cell, providing a second cell, wherein the second cell has a homologous gene of the gene of interest, wherein the homologous gene has been knocked out of the cell, introducing the first construct into the first cell, wherein the first construct is a linear DNA, introducing the second construct into the second cell, wherein the second construct is a linear DNA and evaluating the first and second cell for cell growth in a media, wherein the media lacks an amino acid, wherein the cell growth of the first and second cell are compared to a control cell that has the homologous gene, wherein cell growth of the first and/or second cell is comparable to that of the control cell indicates that the first and second gene of interest encodes a functional protein and wherein cell growth of the first and second cell is slow as compared to the control cell indicates that the first and second gene of interest encodes a non-functional protein or protein with decreased function and indicates that the male and/or female is a carrier of a disease for an amino acid deficiency. In some embodiments, the method further comprises making a diploid strain of a third cell, wherein the third cell comprises the first and second gene of interest and evaluating the third cell for cell growth in a media, wherein the media lacks an amino acid, wherein the cell growth of the third cell are compared to a control cell that has the homologous gene, wherein cell growth of the third cell is comparable to that of the control cell indicates that the first and second gene of interest encodes a functional protein and wherein cell growth of the third cell is slow as compared to the control cell indicates that the first and second gene of interest together indicates a predicted fetus with an amino acid deficiency. In some embodiments, data from the first, second and third cell is stored in a look-up table, wherein the look-up table is generated for disease prediction. In some embodiments, the disease for an amino acid deficiency is NLS.

In a sixth aspect, a method of method of prenatal prediction of an amino acid deficiency wherein at least one parent has a gene mutation is provided. The method comprises the steps: obtaining genomic DNA from a female and male subject, identifying a same gene of interest from the genomic DNA of the female and male subject, wherein the subjects are homozygous or heterozygous for the gene, inserting a first gene of interest from the female into a first construct, inserting a second gene of interest from the male into a second construct, providing a first cell, wherein the first cell has a homologous gene of the gene of interest, wherein the homologous gene has been knocked out of the cell, providing a second cell, wherein the second cell has a homologous gene of the gene of interest, wherein the homologous gene has been knocked out of the cell, making a diploid strain of a third cell, wherein the third cell comprises the first and second gene of interest, introducing the first construct into the first cell, wherein the first construct is a linear DNA, introducing the second construct into the second cell, wherein the second construct is a linear DNA and evaluating the first, second, and third cell for cell growth in a media, wherein the media lacks an amino acid, wherein the cell growth of the first and second cell are compared to a control cell that has the homologous gene, wherein cell growth of the first, second and third cell is comparable to that of the control cell indicates that the first and second gene of interest encodes a functional protein and wherein cell growth of the first, second and third cell is slow as compared to the control cell indicates that the first and second gene of interest encodes a non-functional protein or protein with decreased function and predicts an amino acid deficiency for progeny. In some embodiments, the first gene of interest or the second gene of interest comprises the gene mutation. In some embodiments, the gene of interest encodes PSAT. In some embodiments, the gene of interest encodes PSAT1 with at least one of the amino mutations A99V, S179L, T156M, G78A, R213C or 8222. In some embodiments, data from the first, second and third cell is stored in a look-up table, wherein the look-up table is generated for disease prediction. In some embodiments, the amino acid deficiency is caused by NLS.

In a seventh aspect, a kit for determining amino acid deficiency is provided. The kit comprises a look-up table, wherein the look up table indicates genes or combinations of genes that may be indicative of an amino acid deficiency or disease, one or more nucleic acid probes for detecting one or more mutation in PSAT, wherein the one or more mutation includes at least: G78A, R213C, T156M or R222. In some embodiments, the kit comprises a serine supplement in an amount sufficient to treat a subject suffering from serine deficiency.

In an eighth aspect, a method of determining if a subject is suffering from an amino acid deficiency is provided. The method comprises the steps: providing a look-up table, wherein the look-up table comprises genes or a combination of genes that are indicative of a disease, isolating genes of interest from a subject, determining if the genes include one of more mutations in the look-up table and determining a probability of disease in the subject. In some embodiments, the look up table comprises combination of genes that include genes of PSAT, wherein the PSAT genes encode PSAT comprising mutations G78A, R213C, T156M or 8222. In some embodiments, the look up table comprises a list of mutations that have been determined by the method of claim 43, said method further comprising administering an amino acid supplement to the subject to treat the amino acid deficiency if the subject has more than a 50% probability of having the amino acid deficiency.

In a ninth aspect, a method of treating a subject with an amino acid deficiency is provided. The method comprises the steps: determining if a subject has at least one mutation in PSAT located at G78, R213, T156 or R222 and providing an adequate amount of a serine supplement to the subject if the subject has the at least one mutation. In some embodiments, the at least one mutation is G78A, R213C, T156M or R222, and wherein the subject is an unborn child of a mother, wherein the mother is tested for the presence of the at least one mutation.

In a tenth aspect, a method of identifying a point mutation as a cause or marker of an amino acid deficiency is provided. The method comprises the steps: obtaining genomic DNA from a subject having the amino acid deficiency, identifying a point mutation in the genomic DNA in at least one of 3-PGDH, PSAT1 or PSPH, providing a test yeast cell, wherein a homologous gene of at least one of 3-PGDH, PSAT1 or PSPH has been knocked out of the test yeast cell, introducing the gene with the point mutation into the test yeast cell and evaluating the test yeast cell for cell growth in a media, wherein the media lacks an amino acid, wherein the test yeast cell growth is compared to a control cell that has the homologous gene, wherein when test yeast cell growth is comparable to that of the control cell it indicates that the point mutation still allows for a functional protein, and wherein when the test yeast cell growth is slow as compared to the control cell it indicates that the point mutation results in a non-functional protein or protein with decreased function, thereby identifying the point mutation as a cause or marker of an amino acid deficiency.

In an eleventh aspect, a method of preparing a personalized yeast model for determining a subject at risk of a disease is provided. The method comprises the steps: obtaining genomic DNA from the subject, identifying a gene of interest in at least two alleles from the genomic DNA, inserting the gene into a construct, wherein the construct is a linear DNA, providing a test cell, wherein the test cell has a homologous gene of the gene of interest, wherein the homologous gene has been knocked out of the test cell, introducing the construct into the test cell, evaluating the test cell for cell growth in a media, wherein the media lacks an amino acid, wherein the test cell growth is compared to a control cell that has the homologous gene, wherein when test cell growth is comparable to that of the control cell it indicates that the gene of interest encodes a functional protein, and wherein when the test cell growth is slow as compared to the control cell it indicates that the gene of interest encodes a non-functional protein or protein with decreased function and indicates the disease, generating data for a look-up table; and generating one or more personalized disease amelioration recommendations for the subject; and presenting the one or more personalized disease prevention recommendations for the subject in the personalized disease prevention plan for the subject for disease management. In some embodiments, the method further comprising: a) obtaining a second genomic DNA from a second subject, wherein the second subject has a second set of two alleles that are related to the two alleles of the subject, and second set of two alleles have a second gene of interest, wherein the second gene of interest is placed in a second construct b) introducing the second construct into a second test cell, evaluating the second test cell for cell growth in a media, wherein the media lacks an amino acid, wherein the test cell growth is compared to a control cell that has the homologous gene, wherein when second test cell growth is comparable to that of the control cell it indicates that the gene of interest encodes a functional protein and wherein when the test cell growth is slow as compared to the control cell it indicates that the gene of interest encodes a non-functional protein or protein with decreased function. In some embodiments, the method further comprises obtaining a second genomic DNA from a second subject and identifying a second gene(s) of interest in at least two alleles from the genomic DNA, inserting at least one of the second gene(s) into a second construct, introducing the second construct into a second test cell, and mating the second cell with the first cell to produce a progeny cell. In some embodiments, the method further comprises evaluating the progeny cell for cell growth in a media, wherein the media lacks an amino acid, wherein the test cell growth is compared to a control cell that has the homologous gene, wherein when progeny cell growth is comparable to that of the control cell it indicates that the gene of interest encodes a functional protein, and wherein when the progeny cell growth is slow as compared to the control cell it indicates that the gene of interest encodes a non-functional protein or protein with decreased function and indicates the disease.

### BRIEF DESCRIPTION OF THE DRAWINGS

FIG. 1 shows schematic of the universal cell type for assaying protein function of a protein that is implicated in the serine biosynthesis pathway. As shown, yeast and humans have homologs for proteins implicated in the serine biosynthesis pathway. These enzymes are conserved between yeast and humans.

FIG. 2 shows an exemplary quantitative yeast assay for PSAT1 function in which yeast cells are grown in media that lacks serine. As shown, is a construct of a nucleic acid comprising a transcriptional regulatory sequence with a 5′ and 3′ untranslated regions (UTR) flanking a human coding sequence. In the embodiments herein, the human coding sequence is a homolog of a yeast protein sequence that is implicated in a metabolic pathway. As shown in the bar graph below is the measurement of the growth of yeast cells expressing a control gene (yeast gene SER1), ser1 deletion, PSAT1 gene, PSAT1 variant 1 and PSAT1 variant 2, where variants 1 and 2 are known disease alleles

FIG. 3 shows a quantitative yeast assay for PSAT1 function in the absence of serine. Growth in the absence of exogenously supplied serine, for wildtype strain (“yeast gene”), a ser1 deletion (“no gene”) or strains with human PSAT1 coding sequence or two human disease gene variants (A99V and S179L, respectively). A t-test between “yeast gene” and “human gene” growth was not significant (N.S.) The growth conferred by variants 1 and 2 (disease alleles were significantly below that of the human gene (t-test p-values <0.0001, ***). There was no significant difference between a gene deletion and variant 2, suggesting that variant 2 is equivalent to a complete loss of function.

FIG. 4 shows a comparison between a computational analysis of enzyme mutants and a quantitative yeast assay of the enzyme mutants. As shown, computational methods do a poor job of predicting functional consequences. In the left panel is a bar graph that shows the measurement of the growth of yeast cells expressing several PSAT mutations: 1) 1) K110Q, 2) V149M, 3) T156M, 4) R222*, 5) N236H 6) V250A. As shown in the right panel is a table with the computational predictions of the functions of the PSAT proteins: 1) K110Q, 2) V149M, 3) T156M, 4) R222*, 5) N236H 6) V250A.

FIG. 5 shows an example literature search for several PSAT1 alleles that may be associated with disease. Patients are labeled with the last name of the first author on the publication describing them. Labels refer to the patient identifiers used in those publications, e.g. “patient 1” or “DDD4K.03775”.

FIG. 6 shows a qualitative assay of testing disease alleles individually. Growth in the absence of serine for strains harboring human PSAT1 coding sequence (PSAT1), three known human disease gene variants (A99V, D100A, S179L), or suspected pathogenic variants that were classified as (G78A and R213C). S179L and G78A have growth comparable to null alleles.

FIG. 7 shows an example of an assay for personalized yeast models of affected individuals and carrier parents. Growth in the absence of serine of diploid strains harboring human PSAT1 (PSAT1/PSAT1), the alleles of the carrier parents (PSAT1/A99V or PSAT1/S179L), or the compound heterozygosity of an affected patient from the literature (AV99V/S179L). Constructing and assaying “trios” for all 9 patient genotypes can be performed.

FIG. 8 shows an example of predicting the neural threshold, and pathogenic threshold for the PSAT1 genes. Also calculated is the region between neutral and pathogenic in which pathogenicity cannot be accurately assessed at this time (labeled as variant of uncertain significance (VUS)).

FIG. 9 shows growth values of several haploids in the absence of serine for haploid strains containing the PSAT1 variants listed above each bar. From left to right is PSAT (control), A15V, A99V, c.delG107, D100A, D145Mfs*49, G78A, R213C, R342Dfs*6, S179L, S43R and S43R. N is the number of replicates. Y is the median growth value. NS or asterisks indicate the results of t-tests between a variant and PSAT1 (control). These are all alleles that have been associated with disease in the literature. A15V, A99V, c.delG107, D100A, D145Mfs*49 and S179L are considered in the literature as being pathogenic, G78A, R213C and S43R are annotated as variants of uncertain significance (VUS).

FIG. 10 shows a “personalized yeast model” for affected patients (4) and their carrier parents (2 and 3) (PSAT×D100A and c.delG107×D100A), with the wildtype (1) for reference. Note that these are the same patients that are described in FIG. 5 (Hart et al). The file names list the patient(s), e.g. 01-Hart_1_2 corresponds to Hart 1 and Hart 2 (both have the same parents and genotype). The strains are diploids. As with the previous figures, the PSAT1 variants are listed above each bar. N is the number of replicates. Y is the median growth value. NS or asterisks indicate the results of t-tests between a variant and PSAT1 (carrier, 2 and 3). Note that there are some cases where both parents had the same carrier genotype. In these cases, there is only one bar. As shown in FIG. 10, the progeny has two PSAT alleles (delG107×D100A) that leads to a protein with decreased function.

FIG. 11 shows a “personalized yeast model” for affected patient (3) and their carrier parent(s) (2), with the wildtype (1) for reference. The carrier parents are both Arg342Aspfs*6×PSAT1. Note that these are the same patients that are described in FIG. 5 (Acuna-Hidalgo et al). The strains are diploids. As with the previous figures, the PSAT1 variants are listed above each bar. N is the number of replicates. Y is the median growth value. NS or asterisks indicate the results of t-tests between a variant and PSAT1 (2). Note that there are some cases where both parents had the same carrier genotype. In these cases, there is only one bar. As shown in FIG. 11, the progeny (3) has two PSAT alleles (Arg342Aspfs*6×Arg342Aspfs*6) that leads to a protein with loss of function.

FIG. 12 shows a “personalized yeast model” for affected patient (3) and their carrier parent(s) (2), with the wildtype (1) for reference. Note that these are the same patients that are described in FIG. 5 (Acuna-Hidalgo et al). The strains are diploids. As with the previous figures, the PSAT1 variants are listed above each bar. Both parents are A99V×PSAT1. N is the number of replicates. Y is the median growth value. NS or asterisks indicate the results of t-tests between a variant and PSAT1 (2). Note that there are some cases where both parents had the same carrier genotype, as in this particular case. In these cases, there is only one orange bar. As shown in FIG. 12, the progeny (3) has two PSAT1 alleles (A99V×A99V) that leads to a protein with decreased function.

FIG. 13 shows a “personalized yeast model” for affected patient (3) and their carrier parent(s) (2), with the wildtype (1) for reference. Note that these are the same patients that are described in FIG. 5 (Acuna-Hidalgo et al). The strains are diploids. The parents are both PSAT×S179L. As with the previous figures, the PSAT1 variants are listed above each bar. N is the number of replicates. Y is the median growth value. NS or asterisks indicate the results of t-tests between a variant and PSAT1 (2). Note that there are some cases where both parents had the same carrier genotype, as in this particular case. In these cases, there is only one bar. As shown in FIG. 13, the progeny (3) has two PSAT1 alleles (S179L×S179L) that leads to a protein with complete loss of function.

FIG. 14 shows a “personalized yeast model” for affected patients (4) and their carrier parents (2 and 3), with the wildtype (1) for reference. Note that these are the same patients that are described in FIG. 5 (Acuna-Hidalgo et al). The strains are diploids. The parents are PSAT1×S179L and A99V×PSAT1. As with the previous figures, the PSAT1 variants are listed above each bar. N is the number of replicates. Y is the median growth value. NS or asterisks indicate the results of t-tests between a variant and PSAT1 (carrier, 2 and 3). Note that there are some cases where both parents had the same carrier genotype. In these cases, there is only one bar. As shown in FIG. 14, the progeny (4) has two PSAT alleles (A99V×S179L) that leads to a protein with decreased function.

FIG. 15 shows a “personalized yeast model” for affected patient (3) and their carrier parent(s) (2), with the wildtype (1) for reference. Note that these are the same patients that are described in FIG. 5 (Brassier et al). The strains are diploids. Both parents are S43R×PSAT1. As with the previous figures, the PSAT1 variants are listed above each bar. N is the number of replicates. Y is the median growth value. NS or asterisks indicate the results of t-tests between a variant and PSAT1 (2). Note that there are some cases where both parents had the same carrier genotype, as in this particular case. In these cases, there is only one bar. As shown in FIG. 15, the progeny (3) has two PSAT1 alleles (S43R×S43R) that leads to a protein that is comparatively as functional as the parent.

FIG. 16 shows a “personalized yeast model” for affected patient (3) and their carrier parent(s) (2), with the wildtype (1) for reference. Note that these are the same patients that are described in FIG. 5 (Monies et al). The strains are diploids. Both parents are G78A×PSAT1. As with the previous figures, the PSAT1 variants are listed above each bar. N is the number of replicates. Y is the median growth value. NS or asterisks indicate the results of t-tests between a variant and PSAT1 (2). Note that there are some cases where both parents had the same carrier genotype, as in this particular case. In these cases, there is only one orange bar. As shown in FIG. 16, the progeny (3) has two PSAT1 alleles (G78A×G78A) that leads to a protein has a complete loss of function as compared to the parent and the wild type.

FIG. 17 shows a “personalized yeast model” for affected patient (3) and their carrier parent(s) (2), with the wildtype (1) for reference. Note that these are the same patients that are described in FIG. 5 (McRae et al). The strains are diploids. Both parents are R213C×PSAT1. As with the previous figures, the PSAT1 variants are listed above each bar. N is the number of replicates. Y is the median growth value. NS or asterisks indicate the results of t-tests between a variant and PSAT1 (2). Note that there are some cases where both parents had the same carrier genotype, as in this particular case. In these cases, there is only one orange bar. As shown in FIG. 17, the progeny (3) has two PSAT1 alleles (R213C×R213C) that leads to a protein has decreased function as compared to the parent and the wild type.

FIG. 18 shows a “personalized yeast model” for affected patients (4) and their carrier parents (2 and 3), with the wildtype (1) for reference. Note that these are the same patients that are described in FIG. 5 (Glinton et al). The strains are diploids. The parents are PSAT1×A15V and D145Mfs*49×PSAT100). As with the previous figures, the PSAT1 variants are listed above each bar. N is the number of replicates. Y is the median growth value. NS or asterisks indicate the results of t-tests between a variant and PSAT1 (carrier, 2 and 3). Note that there are some cases where both parents had the same carrier genotype. In these cases, there is only one bar. As shown in FIG. 18, the progeny (4) has two PSAT alleles (D145Mfs*49×A15V) that lead to a protein with decreased function as compared to the wild type. However, the progeny does have similar protein characteristics with one of the parents (Bar 3).

### DEFINITIONS

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

“Polynucleotide,” as described herein refers to “nucleic acid” or “nucleic acid molecule,” such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g, enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some alternatives, a nucleic acid sequence encoding a fusion protein is provided. In some alternatives, the nucleic acid is RNA or DNA.

“Expression construct” or “construct” is a nucleic acid used to introduce heterologous nucleic acids into a cell that has regulatory elements to provide expression of the heterologous nucleic acids in the cell. The constructs described herein include but are not limited to plasmid, minicircles, yeast, and viral genomes.

A single copy of the construct is inserted stably into the yeast genome (at either the same position as the yeast gene, which has been deleted or at a neutral position in the genome, e.g. the HO locus). In the embodiments herein, the construct is a “linear DNA molecule (double or single stranded) that can be integrated into the yeast nuclear or mitochondrial genome by homologous recombination. These constructs may be synthesized in vitro by a commercial gene synthesis company (e.g. Twist Biosciences or IDT). As such the nucleic acid sequence could be the human sequence or a yeast optimized version that encodes the same protein sequence.

“Coding for” or “encoding” have their plain and ordinary meaning when read in light of the specification, and may include but is not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

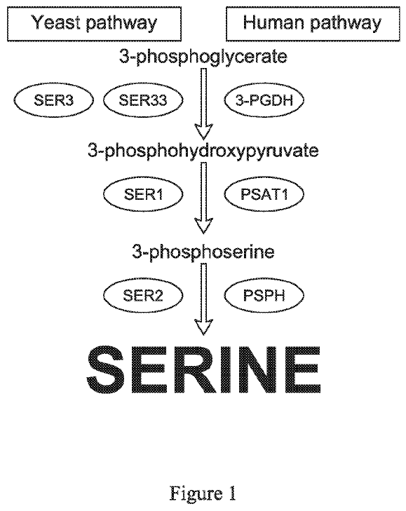

Serine Deficiency Syndrome/Neu-Laxova Syndrome (NLS) refers to a disease of individuals with homozygous or compound heterozygous loss of function mutations in any of three metabolic genes responsible for the biosynthesis of serine cause a severe neurodevelopmental syndrome that presents with congenital microcephaly, intractable seizures, and severe psychomotor retardation (See FIG. 1). As described herein, Serine Deficiency Syndrome and Neu-Laxova Syndrome (NLS) are used interchangeably. Neu-Laxova syndrome (NLS) is a very severe disorder, leading to stillbirth, neonatal death, or severe developmental delay. However, if diagnosed early, dietary supplementation can reduce or even eliminate disease symptoms. In one case study, prenatal diagnosis followed by serine supplementation (to the mother) starting early in pregnancy and to the patient from birth onward completely prevented the onset of symptoms. Serine sources can be taken at a 600/mg/kg/day for those suffering from a serine deficiency in 4 to 6 doses in a day.

NLS is a heterogeneous metabolic disorder caused by homozygous or compound heterozygous mutations in the PHGDH, PSAT1 and PSPH genes, which are involved in the serine biosynthesis pathway and are essential for cell proliferation. Mutations in all three genes had been previously identified as the cause of serine-deficiency syndromes. Milder forms of the disease also exist. NLS is found in 1 in 5000 births.

“Retinal neuropathy” is any damage to the retina of the eyes, which may cause vision impairment. Retinopathy often refers to retinal vascular disease, or damage to the retina caused by abnormal blood flow. Age-related macular degeneration is technically included under the umbrella term retinopathy but is often discussed as a separate entity. Retinopathy, or retinal vascular disease, can be broadly categorized into proliferative and non-proliferative types. In some cases the retinal neuropathy may be caused by a deficiency in an amino acid, such as serine. In some embodiments herein, a subject is at risk at developing retinal neuropathy and is selected to receive an appropriate amount of serine for treatment.

Additionally, milder forms of serine deficiencies have been reported. These milder forms of serine deficiencies have been predicted to be associated with adult onset diseases. Without being limiting, several diseases may be (We refer to retinal disease a few times, but it would be good to introduce the fact that milder forms of serine deficiency (or other amino acid disorders) might be associated with adult onset diseases. An example is in Scerri et al. (Nat Genet. 2017 April; 49(4):559-567; incorporated by reference in its entirety herein). Scerri et al. describes a serine deficiency, macular telangiectasia type 2 (MacTel), which manifests at age 40-60, in which subjects afflicted by MacTel suffer from abnormal right-angled juxtafoveolar capillaries and parafoveal telangiectasias. For MacTel, mutations in several alleles have been implicated in the disease which leads to problems in the glycine and serine metabolic pathway.

The subject as described in the embodiments herein may be a fetus, neonate, juvenile or adult. “Fetus” has its plain and ordinary meaning when read in light of the specification, and may include but is not limited to, for example, a prenatal human between its embryonic state and its birth. “Neonate” refers to a newborn child. “Juvenile” refers to a person under the established age of 18 years. “Adult” refers to a person any age over 18.

“Look-up table” has its plain and ordinary meaning when read in light of the specification, and may include but is not limited to, for example, a table prepared with a is a combination of genes that may predict a fatal outcome. In the embodiments herein, the look-up table may predict a retinal disease dependent on serine, NLS, or metabolic disease such as an amino acid deficiency. The data on the look-up table also provide different predicted thresholds for determining the functions of the protein as there are different thresholds for combinations of alleles for different diseases.

“Cell growth” also referred to as the “growth value”, is measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the aforementioned parameters.

### DETAILED DESCRIPTION

In humans, serine is a nutritionally non-essential amino acid. The main source of essential amino acids is from the diet, however non-essential amino acids are normally synthesized by humans and other mammals from common intermediates. As shown in FIG. 1, serine is biosynthesized from a glycolytic intermediate, 3-phosphoglycerate (3-PG), in a three-step process involving the enzymes: 3-phosphoglycerate dihydrogenase (3-PGDH), phosphoserine aminotransferase (PSAT), and phosphoserine phosphatase (PSP).

Serine can also be derived in a reversible reaction from glycine; through degradation of protein and phospholipids; and through dietary intake. For this reason, NLS is usually treated by dietary supplementation with both serine and glycine. The primary pathway maintaining adequate serine concentrations is likely to depend on tissue type and stage of development as described by de Koning et al., 2003 (de Koning et al., 2003).

Serine plays a functional role in cell growth and development. For example, the conversion of serine to glycine by serine hydroxymethyltransferase results in the formation of the one-carbon units necessary for the synthesis of the purine bases, adenine and guanine. These bases may be linked to the phosphate ester of pentose sugars and are essential components of DNA and RNA, with end-products of energy producing metabolic pathways, ATP and GTP. Additionally, serine conversion to glycine through the enzymatic pathway provides one-carbon units necessary for production of the pyrimidine nucleotide, deoxythymidine monophosphate, which is also an essential component of DNA.

Serine also plays a role as a precursor for the neurotransmitters glycine and D-serine, and indirectly through L-cysteine, for the neurotransmitter taurine. Glycine can be synthesized from serine and is an inhibitory neurotransmitter, which may bind to the glycine receptor on the post-synaptic membrane or together with glutamate as a co-agonist. Essential components that can be made from serine include D-serine, which is an agonist of the NMDA receptor and cysteine, which can be formed from serine through a trans-sulfuration pathway. This leads to precursors for proteins, glutathione, taurine, coenzyme A and inorganic sulfate. Taurine functions as an inhibitory neurotransmitter and is likely to play a role in pre- and post-natal development of the central nervous system. Thus, serine is much needed in multiple pathways such as cell growth pathways.

Serine deficiency is a rare, inherited, metabolic disorder of serine biosynthesis. The majority of the cases reported in the literature so far show a decrease in 3-phosphoglycerate dehydrogenase (3-PGDH) activity resulting in low fasting serum and CSF serine levels. Children with serine deficiency present with congenital microcephaly may develop severe psychomotor retardation and intractable seizures.

Several mutations have been identified in the gene encoding human 3-PGDH located on chromosome one (1p12 or 1q12). Several examples of mutations are described in Klomp et al, 2000 and Pind et al, 2002. Some mutations result in a substitution of valine for methionine at position 490 (V490M) of the enzyme in most of the cases reported, or a V425M substitution in one reported case (Klomp et al, 2000, Pind et al, 2002).

Nearly all children born with serine deficiency have congenital microcephaly. In addition, hallmark signs of Serine deficiency include severe psychomotor retardation, seizures, spastic quadriplegia and in some patients, nystagmus, megaloblastic anemia, cataract and hypogonadism.

In children born with serine deficiency, a low level of serine in the cerebral spinal fluid (CSF) is the most reliable indicator for both 3-phosphoglycerate dehydrogenase (3-PGDH) and 3-phosphoserine phosphatase (3-PSP) deficiency. In some, but not all cases, CSF glycine levels were below normal. In the fasted state, plasma serine and to a lesser extent, plasma glycine levels are below normal levels. Urine amino acid levels are normal. (de Koning and Klomp. 2004). Individuals with homozygous or compound heterozygous loss of function mutations in any of three metabolic genes responsible for the biosynthesis of serine may cause a severe neurodevelopmental syndrome that presents with congenital microcephaly, intractable seizures, and severe psychomotor retardation. Thus a mutation leading to an enzyme with a loss of function would lead to the limiting step of serine biosynthesis.

The currently accepted diagnostic for serine deficiency is measuring serine levels from the cerebrospinal fluid, which cannot be performed until after birth when many of the symptoms, such as developmental delay, are irreversible. As such, a method is needed to determine the likelihood of developing the disease in a fetus in order to provide treatment prior to the manifestation of the disease. By determining which alleles of the serine biosynthetic genes are neutral or pathogenic, the assays described would facilitate a gene-sequencing based diagnostic that can be used earlier (including on the developing fetus or the asymptomatic parents)

### Development of an Assay for Determining Deleterious Proteins

As provided herein, an assay was developed to determine a deleterious protein that functions in a metabolic pathway. As shown in FIG. 1, serine is produce in a three step pathway to generate serine from 3-phosphoglycerate. In humans, this relies on three proteins 3-PGDH, PSAT1 and PSPH which are also conserved homologs in yeast (SER3, SER33, SER1 and SER2). It has been shown that PSAT1 can be knocked out and replaced in yeast with the human homolog. It is anticipated that knockouts of the 3-PGD-H and PSPH can be replaced by their human homologs as well. Moreover, as provided herein, this modified yeast can then be used as a model organism for further testing.

A construct encoding the human variant can be used for expression in a yeast system that has been knocked out of the yeast homolog. For example, the yeast can be genetically engineered to have the gene SER1 knocked out. The yeast is then genetically engineered to express PSAT1 from a nucleic acid as shown in FIG. 1. Expression constructs for yeast are known by those of skill in the art and may have a strong yeast promoter/terminator and may comprise a yeast selectable marker cassette.

An assay can then be run to determine the yeast cells growth in comparison to a yeast cell expressing the wild type SER1 or a yeast cell expressing a PSAT1 gene that has no mutations. The biochemical pathway that produces serine is highly conserved between humans and yeast so as to allow yeast to adequately serve as a model organism. Loss of function mutations in any of the three serine biosynthetic genes in humans cause Serine Deficiency Syndrome. In yeast, loss of function of the yeast enzymes produces a quantitative and easily measured trait, poor growth in the absence of serine supplementation.

### Kits and Systems

Also provided herein are kits and systems including the cells, expression constructs, and protein sequences provided and described herein as well as written instructions for making and using the same. Thus, for example, provided herein is a kit comprising one or more of: a protein sequence as described herein; an expression construct as described herein; and/or a cell as described herein. Also provided is a system for preparing diploid stains for assaying combinations of genes in a cell as described herein, wherein the cell comprises an expression construct, wherein the construct comprises a nucleic acid encoding a protein sequence as described herein.

A look up table is also provided for use with the system to assess the combination of genes that may indicate deleterious combinations. The look-up table is a table that correlates one form of data to another form, or one or more forms of data to a predicted outcome to which the data is relevant, such as phenotype or trait. For example, a look-up table can comprise a correlation between allelic data for at least one protein that is involved in a metabolic pathway and a particular trait or phenotype, such as a particular disease diagnosis, that an individual who comprises the particular allelic data is likely to display, or is more likely to display than individuals who do not comprise the particular allelic data. Look-up tables can be multidimensional, for example, without being limiting, they can contain information about multiple alleles for a particular protein or proteins simultaneously, or they can contain information about multiple protein variants, such as mutational variants, and they may also comprise other factors, such as particulars about diseases diagnoses, therapeutic methods or drugs to be used with a particular disease. In some embodiments, the look-up table comprises correlations between at least one allele and a disease. In some embodiments, the look-up table comprises a correlation for a plurality of diseases. In all scenarios, by referencing to a look-up table that gives an indication of a correlation between an allele and a metabolic disease, such as a serine deficiency, a disease or predicted progeny can be identified in the individual from whom the sample is derived. In some embodiments, the correlation is reported as a statistical measure. The statistical measure may be reported as a risk measure, such as a relative risk (RR), an absolute risk (AR) or an odds ratio (OR). They can also be reported as a having a pathogenic threshold for the protein, neural threshold or as a variable unknown.

A look-up table may be generated by the individual assessment of alleles or by studies of diploid cells that are made by crossing cells that are homozygous for a normal gene and cells that cells that have the mutated from of the gene that is to be tested.

Kits can be used that provide a look up table, wherein the look up table indicates genes or combinations of genes that may be indicative of an amino acid deficiency or disease and one or more nucleic acid probes for detecting one or more mutation in PSAT, wherein the one or more mutation includes at least: G78A, R213C, T156M, R222. In some embodiments, the kit comprises a supplement. In some embodiments, the supplement is serine that is in an amount sufficient to treat a subject suffering from serine deficiency.

### Example 1. Assays for Determination of PSAT Variant Function

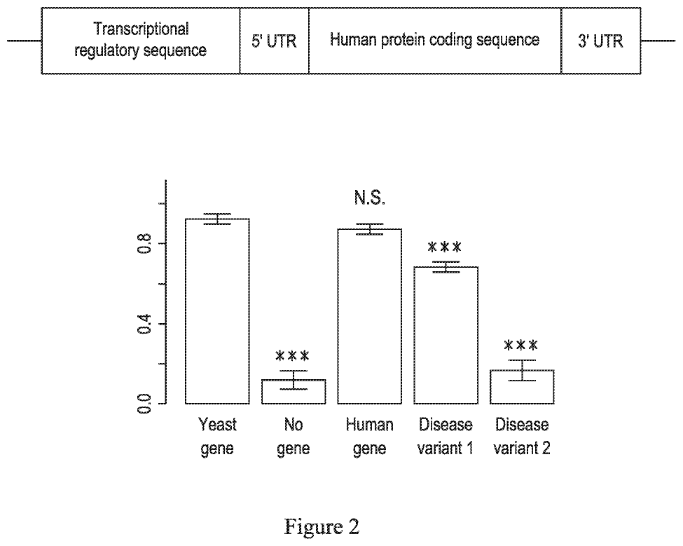

As shown in FIG. 2, the human protein coding sequence of the PSAT1 gene can functionally replace the corresponding SER1 gene in yeast. This single copy can be stably integrated into the genome under the control of the native yeast regulatory sequences for transcription, translation, and mRNA stability with the nucleic acid comprising the sequence as shown in FIG. 2.

The PSAT1 sequence was codon optimized for expression in yeast (SEQ ID NO: 1:

ATGGACGCGCCAAGACAGGTTGTGAATTTCGGACCTGGGCCAGCTAAATT
GCCTCACTCTGTTTTGTTGGAAATCCAGAAAGAGTTATTAGATTATAAAG
GTGTAGGTATATCAGTCTTGGAAATGTCTCACCGTTCGTCTGATTTCGCC
AAAATAATTAATAACACCGAGAACTTAGTTAGGGAACTGCTAGCCGTTCC
TGACAACTATAAAGTGATTTTTTTGCAAGGTGGTGGCTGCGGTCAATTTT
CTGCCGTTCCTTTAAATTTGATAGGTTTAAAAGCGGGTAGATGTGCCGAC
TATGTCGTCACCGGTGCGTGGTCTGCTAAAGCCGCTGAAGAGGCAAAGAA
ATTCGGTACTATCAACATTGTTCATCCAAAGTTGGGCTCTTACACTAAAA
TTCCGGATCCATCCACATGGAATTTAAATCCCGATGCATCATACGTGTAC
TACTGTGCTAATGAAACTGTTCATGGTGTTGAATTTGACTTTATTCCAGA
TGTTAAGGGTGCTGTTTTGGTTTGCGACATGTCCAGCAACTTCTTGTCTA
AGCCAGTTGACGTTTCTAAATTCGGTGTCATCTTTGCAGGCGCCCAAAAA
AACGTTGGTTCTGCAGGCGTAACCGTGGTTATTGTCAGAGATGACTTACT
TGGTTTTGCGTTAAGAGAATGCCCTTCTGTTTTAGAGTATAAGGTGCAGG
CCGGTAACTCATCACTGTATAATACACCACCATGCTTCTCTATATATGTT
ATGGGTCTCGTGTTAGAATGGATTAAGAATAATGGTGGTGCAGCTGCGAT
GGAGAAATTGTCTTCTATTAAGTCTCAGACGATATATGAGATAATAGATA
ACTCACAGGGTTTTTACGTTTGCCCAGTGGAACCACAGAATAGATCAAAA
ATGAATATCCCATTCCGTATAGGTAACGCGAAGGGCGATGACGCCCTTGA
AAAGAGGTTTCTAGATAAGGCACTTGAGTTAAATATGCTGTCTTTGAAAG
GACATCGTTCAGTTGGTGGGATCAGAGCCTCCTTGTATAACGCGGTGACG
ATCGAAGACGTACAAAAGTTGGCCGCTTTTATGAAGAAATTTTTGGAGAT
GCACCAGTTATAA)

For frameshift mutations an optimized sequence was used up to the mutation. After the SNP, the human cDNA sequence, SEQ ID NO: 2, was used. (SEQ ID NO: 2:

ATGGACGCCCCCAGGCAGGTGGTCAACTTTGGGCCTGGTCCCGCCAAGCT
GCCGCACTCAGTGTTGTTAGAGATACAAAAGGAATTATTAGACTACAAAG
GAGTTGGCATTAGTGTTCTTGAAATGAGTCACAGGTCATCAGATTTTGCC
AAGATTATTAACAATACAGAGAATCTTGTGCGGGAATTGCTAGCTGTTCC
AGACAACTATAAGGTGATTTTTCTGCAAGGAGGTGGGTGCGGCCAGTTCA
GTGCTGTCCCCTTAAACCTCATTGGCTTGAAAGCAGGAAGGTGTGCGGAC
TATGTGGTGACAGGAGCTTGGTCAGCTAAGGCCGCAGAAGAAGCCAAGAA
GTTTGGGACTATAAATATCGTTCACCCTAAACTTGGGAGTTATACAAAAA
TTCCAGATCCAAGCACCTGGAACCTCAACCCAGATGCCTCCTACGTGTAT
TATTGCGCAAATGAGACGGTGCATGGTGTGGAGTTTGACTTTATACCCGA
TGTCAAGGGAGCAGTACTGGTTTGTGACATGTCCTCAAACTTCCTGTCCA
AGCCAGTGGATGTTTCCAAGTTTGGTGTGATTTTTGCTGGTGCCCAGAAG
AATGTTGGCTCTGCTGGGGTCACCGTGGTGATTGTCCGTGATGACCTGCT
GGGGTTTGCCCTCCGAGAGTGCCCCTCGGTCCTGGAATACAAGGTGCAGG
CTGGAAACAGCTCCTTGTACAACACGCCTCCATGTTTCAGCATCTACGTC
ATGGGCTTGGTTCTGGAGTGGATTAAAAACAATGGAGGTGCCGCGGCCAT
GGAGAAGCTTAGCTCCATCAAATCTCAAACAATTTATGAGATTATTGATA
ATTCTCAAGGATTCTACGTTTGTCCAGTGGAGCCCCAAAATAGAAGCAAG
ATGAATATTCCATTCCGCATTGGCAATGCCAAAGGAGATGATGCTTTAGA
AAAAAGATTTCTTGATAAAGCTCTTGAACTCAATATGTTGTCCTTGAAAG
GGCATAGGTCTGTGGGAGGCATCCGGGCCTCTCTGTATAATGCTGTCACA
ATTGAAGACGTTCAGAAGCTGGCCGCCTTCATGAAAAAATTTTTGGAGAT
GCATCAGCTATGA) (PSAT1 cDNA Genbank Accession
number: CV023706)

In some embodiments, the PSAT1 is encoded by a gene comprising a sequence set forth in SEQ ID NO: 3.

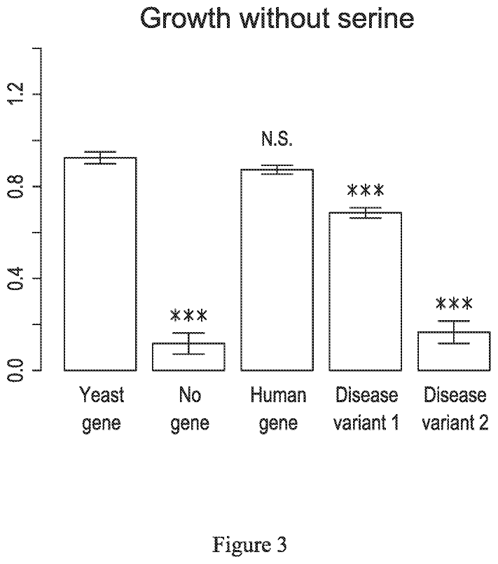

Cells were grown in liquid rich yeast medium (YPD) broth that contains serine. Cells expressing the wild type SER1 (yeast homolog of PSAT1), no gene, PSAT1, PSAT1 mutant A99V, PSAT1 mutant S179L, were used to inoculate (by replica pinning) prepared solid agar plates containing yeast minimal medium (SD), which lacks serine. Growth of each strain, which contains a PSAT1 variant or a control (e.g. the yeast SER1 gene or a ser1 deletion), was quantified by photographing the yeast growing on the solid agar plates and performing image analysis using custom software (written by the inventors). The assays include technical and biological replicates. As shown, the loss of function of the yeast enzymes produces a quantitative and easily measured trait, poor growth in the absence of serine supplementation. In the example below, we show that the human protein coding sequence of the PSAT1 gene can functionally replace the corresponding SER1 gene in yeast. This gives a yeast-based assay for measuring the activity of PSAT1 gene variants found in a specific patient, a genome-wide association study (GWAS), or the population at large. (FIG. 3). As shown, is the growth in the absence of serine, for wildtype strain (“yeast gene”), a SER1 deletion (“no gene”) or strains with human PSAT1 coding sequence or two human disease gene variants (A99V and S179L, respectively). A t-test between “yeast gene” and “human gene” growth was not significant (N.S.) The growth conferred by variants 1 and 2 (disease alleles were significantly below that of the human gene (t-test p-values <0.0001, ***). There was no significant difference between a gene deletion and variant 2, suggesting that variant 2 is equivalent to a complete loss of function. The tests on the yeast strains may be performed on either agar plates or in a liquid media.

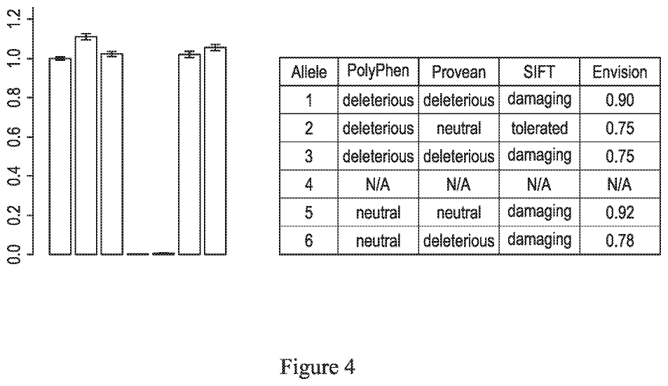

Assays were performed to assess the function of genes that were determined by computational methods to be either functional or non-functional (FIG. 4). Tests were performed on PSAT proteins with the mutations: 1) K110Q, 2) V149M, 3) T156M, 4) R222*, 5) N236H 6) V250A. As shown in the table (FIG. 4, right panel), PSAT proteins with the mutations K110Q and V149M were determined to be deleterious proteins. PSAT proteins with the mutations N236H and V250A were considered to be “neutral” while T156M and 8222 were predicted to be deleterious or with an undetermined function, respectively.

Assays for PSAT1 protein functions were performed on PSAT1 proteins with the mutations 1) K110Q, 2) V149M, 3) T156M, 4) 8222, 5) N236H 6) V250A to determine protein function as compared to the predicted protein characteristics as determined by the computational methods (FIG. 4, right panel). As shown in the left panel of FIG. 4, the mutants K110Q, V149M, N236H and V250A were shown to have growth that was comparable to the growth of the wild type PSAT1 protein. The PSAT proteins with the mutations T156M and 8222 were shown to be deleterious by this method. Thus the assay provides a fast diagnostic to show the functionality of a protein which cannot be predicted by a computational analysis.

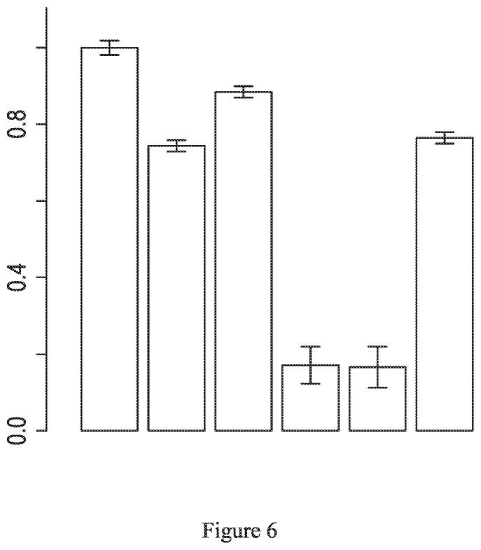

The assay can be used to perform tests on individual alleles as well. As shown in FIG. 6, additional mutants were also tested for their performance in media that was lacking in serine. As shown, the PSAT mutants with a G78A and R213C mutation were compared to the growth of cells expressing PSAT1 (control), A99V, D100, and 5179. These proteins were previously predicted to be variable unknowns. As shown is the growth data of the three known human disease gene variants (A99V, D100A and S179L), and suspected pathogenic variants that were classified as variable outcome strains (S179L and G78A). As shown, the S179L and G78A mutants have growth comparable to null alleles.

Cell growth, may also be referred to as the “growth value”, and may be measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the aforementioned parameters.

### Example 2: Assays for Personalized Yeast Models for Affected Individuals and Carriers

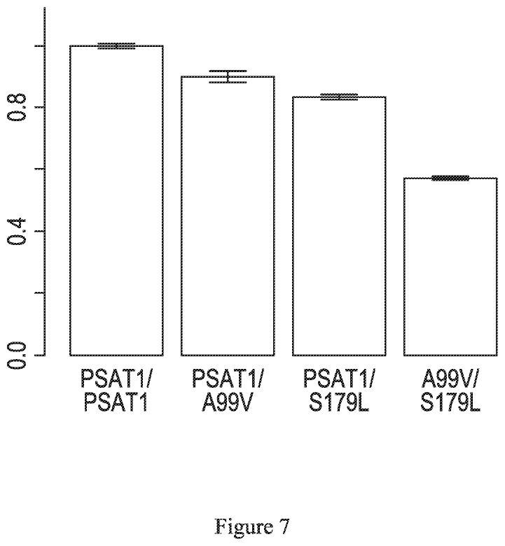

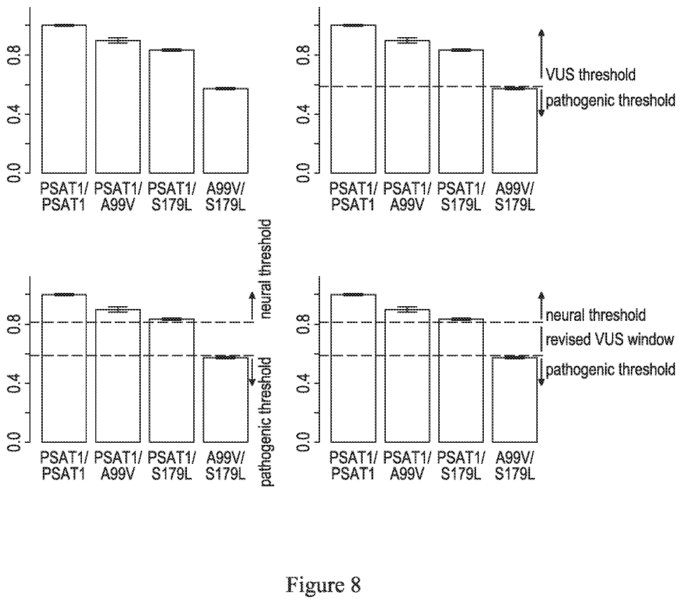

Diploid strains carrying PSAT1/PSAT1, PSAT1/A99V and PSAT1/S179L were prepared. These diploid strains were prepared by mating haploid cells each containing one of the two variants, e.g. A99V and S179L. The resulting diploid cell is heterozygous for the two variants. Homozygous diploid strains and strains that are heterozygous for a wildtype and mutant version of the gene, e.g. PSAT1/A99V, are made in the same way. The yeast cells were then analyzed for growth in YPD media that lacked serine. This test may also be performed in agar plates or liquid media for assessment of cell growth. As shown in FIG. 8, the diploid cells PSAT1/A99V and PSAT1/S179L were shown to function above the pathogenic threshold, which indicates that the protein is predicted to be functional, however the proteins have reduced activity as compared with the PSAT1/PSAT1 homozygous. The pathogenic threshold may be calculated for different heterozygotes. Crossing cells to generate a heterozygous A99V/S179L was performed to test the heterozygote for growth in media lacking serine. As shown in the graph, the heterozygous A99V/S179L produced a PSAT1 protein with decreased function which was predicted to lead to a pathogenic phenotype in a progeny carrying both the A99V and the S179L genes. As such, the data generated from the assays may be used to determine a threshold for disease and may be used to create personalized data prediction of disease, prediction of adult onset of a disease and prediction of disease of progeny for two subjects. An example of a personalized test is shown in FIG. 7. As shown in FIG. 7, is a test of diploid strains. The control is homozygous for PSAT1. The two parents are heterozygous for PSAT1/A99V and PSAT1/179L. As shown the progeny is a carrier of the A99V and the S179L mutation. The protein expressed by the progeny has decreased function as compared to the parent strains as well as the control cell.

Cell growth, may also be referred to as the “growth value”, and may be measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the aforementioned parameters.

### Example 3: Assessment of Diploid Yeast Strains Carrying PSAT Mutations for Personalized Model Systems

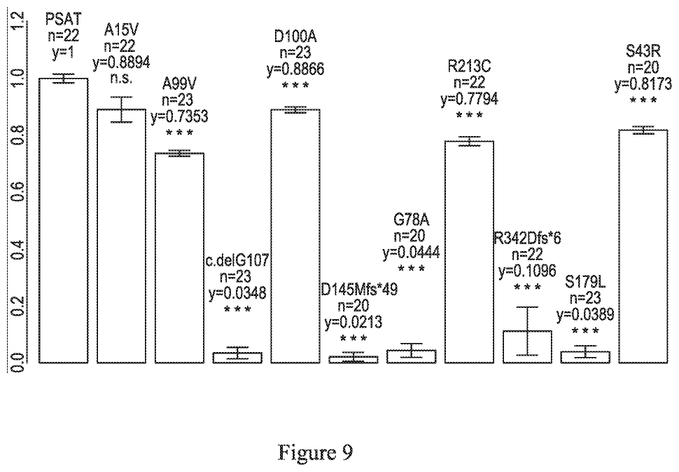

As shown in FIG. 9, the growth values of several haploids in the absence of serine for haploid strains containing the PSAT1 variants listed above each bar. The cells are grown in the methods previously described in which the media is lacking the essential amino acid, serine. From left to right is PSAT (control), A15V, A99V, c.delG107, D100A, D145Mfs*49, G78A, R213C, R342Dfs*6, S179L, S43R and S43R. N is the number of replicates. Y is the median growth value. NS or asterisks indicate the results of t-tests between a variant and PSAT1 (control). These are all alleles that have been associated with disease in the literature. A15V, A99V, c.delG107, D100A, D145Mfs*49 and S179L are considered in the literature as being pathogenic, G78A, R213C and S43R are annotated as variants of uncertain significance (VUS). As shown, the haploid cells carrying the mutant PSAT1 gene have a decreased function, with the significant lack of function is shown for mutants c.delG107, D145Mfs*49, G78A, R342Dfs*6 and S179L.

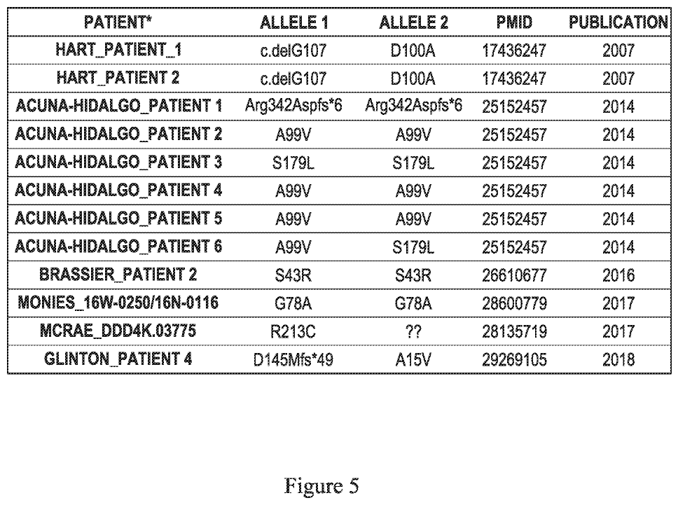

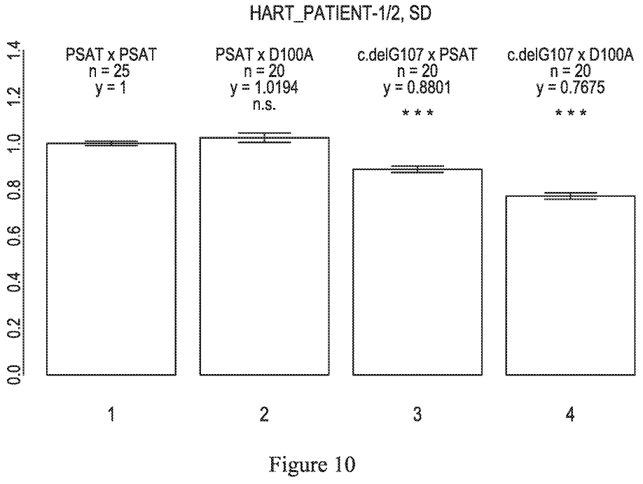

Tests were then performed on diploid strains in order to examine the progeny cells that may carry at least one mutation in a diploid strain. As shown in FIGS. 10-18, the mutants as described in FIG. 5, were used to examine the effects of having either one or two recessive genes that were shown to effect the growth of cells in a serine-free media.

As shown in FIG. 10, is a personalized yeast model for an affected patient and carrier parents (PSAT×D100A and c.delG107×D100A). As shown in FIG. 10, the progeny has two PSAT alleles (delG107×D100A) that leads to a protein with decreased function. Thus a patient with this genotype, may need to be provided with a therapy that involves supplementation of the amino acid in order to have normal cellular functions.

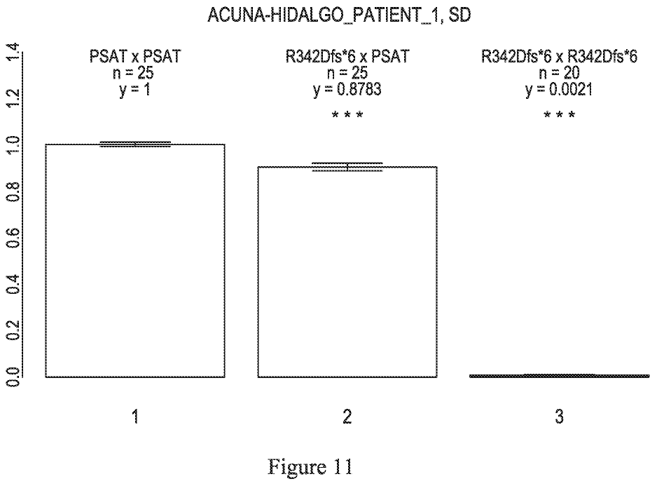

As shown in FIG. 11, is a personalized yeast model for an affected patient with the carrier parents that both have the allele for a wild type PSAT1 gene and Arg342Aspfs gene. As shown, the progeny (3) has two PSAT alleles (Arg342Aspfs*6×Arg342Aspfs*6) that leads to a protein with loss of function. Thus a patient with this genotype, may need to be provided with a therapy that involves supplementation of the amino acid in order to have normal cellular functions. As this test may be performed on individuals who are expecting, Determining a fetus with this genotype, one may need to provide with a therapy for a pregnant subject that involves supplementation of the amino acid in order to have normal cellular functions in a fetus.

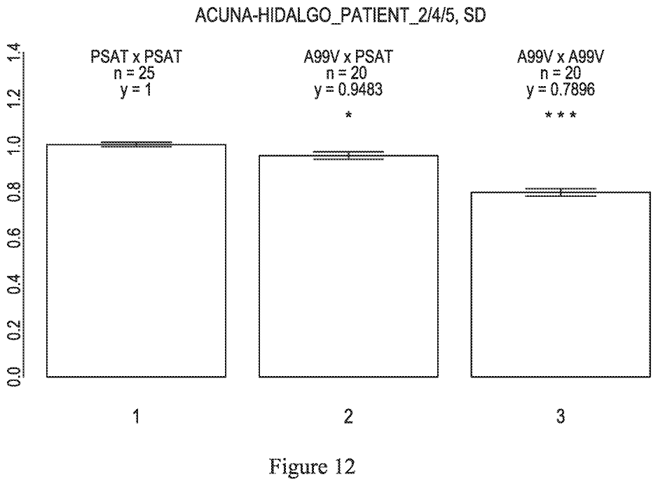

As shown in FIG. 12, is a personalized yeast model for an affected patient with the carrier parents that both have the allele for A99V and the wild type PSAT1 gene. As shown, the progeny (3) has two PSAT1 alleles (A99V×A99V) that leads to a protein with decreased function. Thus a patient with this genotype, may need to be provided with a therapy that involves supplementation of the amino acid in order to have normal cellular functions.

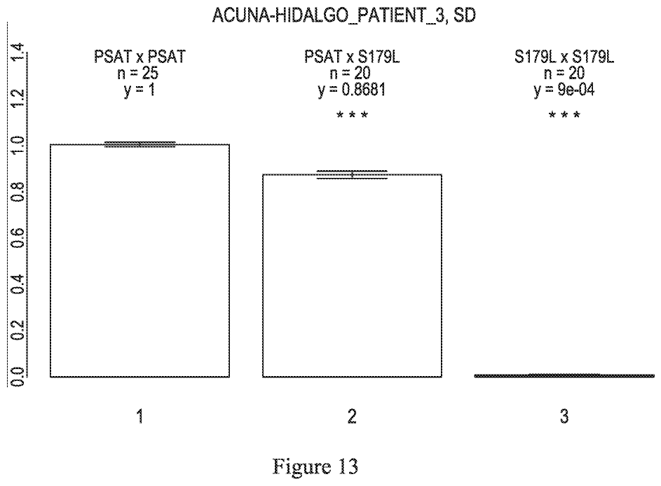

As shown in FIG. 13, is a personalized yeast model for an affected patient with the carrier parents that both have the allele PSAT1 gene and gene for the PSAT1 with the S179L mutation. As shown in FIG. 13, the progeny (3) has two PSAT1 alleles (S179L×S179L) that leads to a protein with complete loss of function. As this test may be performed on individuals who are expecting, Determining a fetus with this genotype, one may need to provide with a therapy for a pregnant subject that involves supplementation of the amino acid in order to have normal cellular functions in a fetus. This test may also be performed for genetic testing for a couple in order to determine their probability of having a child with a metabolic disease.

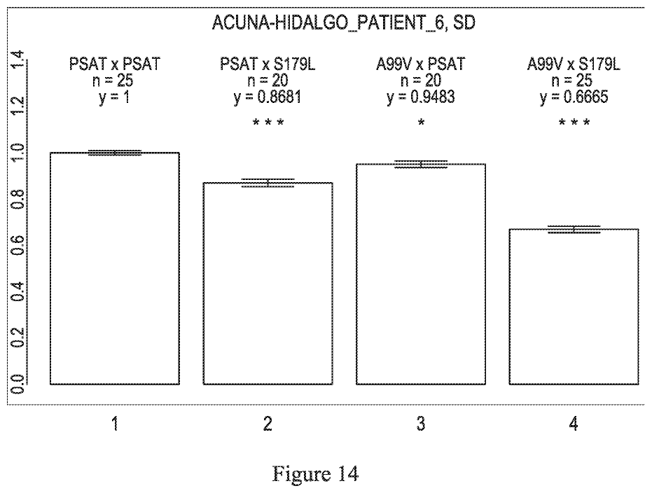

As shown in FIG. 14, is a personalized yeast model for an affected patient with the carrier parents that both are heterozygous for the wild type PSAT1 and a recessive PSAT1 mutant gene (PSAT1×S179L and A99V×PSAT1). As shown, the progeny (4) has two PSAT alleles (A99V×S179L) that leads to a protein with decreased function. Thus a patient with this genotype, may need to be provided with a therapy that involves supplementation of the amino acid in order to have normal cellular functions.

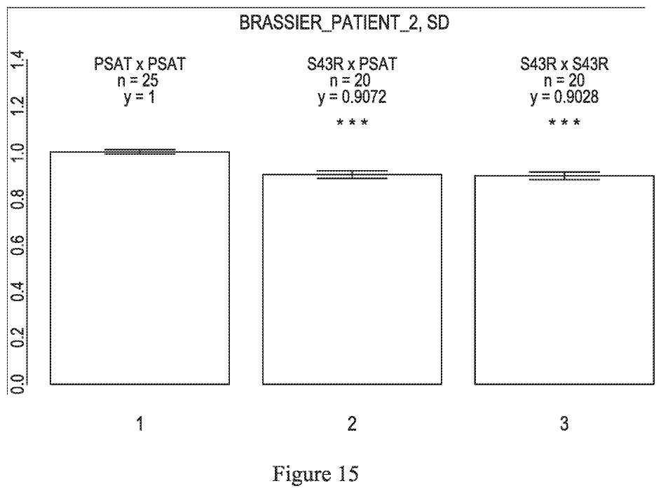

As shown in FIG. 15, is a personalized yeast model for an affected patient with the carrier parents that both are heterozygous for the wild type PSAT1 and a recessive PSAT1 mutant gene. (both parents: S43R×PSAT1). As shown in FIG. 15, the progeny (3) has two PSAT1 alleles (S43R×S43R) that leads to a protein that's comparatively as functional as the parent. Thus assessment of the health of the patient will need to be evaluated to determine if serine supplementation is needed.

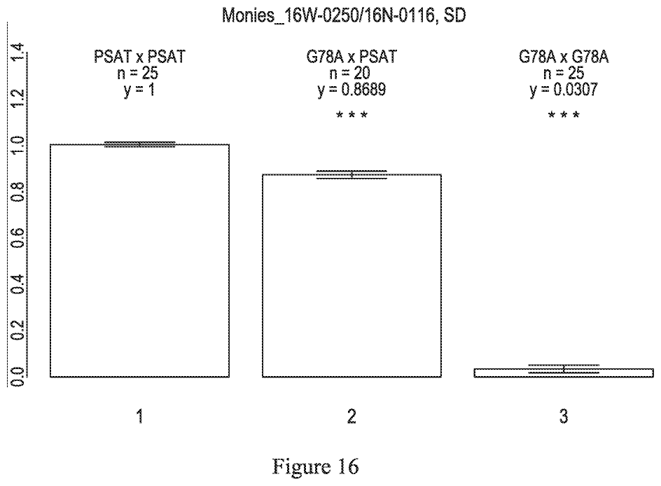

A personalized yeast model was also performed for parents that have the alleles for PSAT1 with the G78A mutation and the wild type PSAT1. As shown in FIG. 16, the progeny (3) has two PSAT1 alleles (G78A×G78A) that leads to a protein has a complete loss of function as compared to the parent and the wild type.

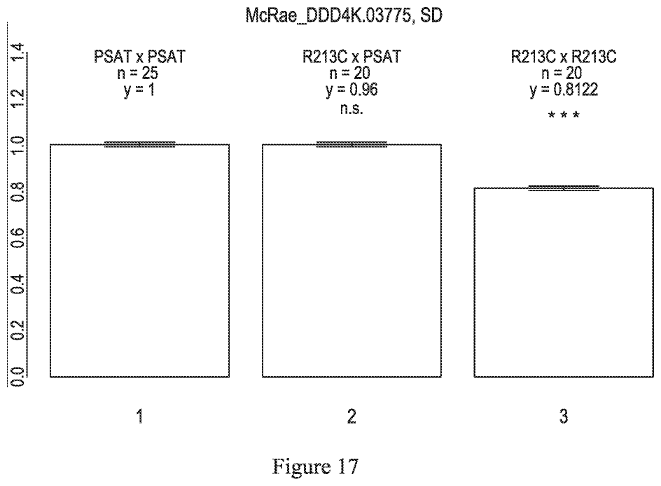

A personalized yeast model was also performed for parents that have the alleles for PSAT1 with the R213C mutation and the wild type PSAT1. As shown in FIG. 17, the progeny (3) has two PSAT1 alleles (R213C×R213C) that leads to a protein has decreased function as compared to the parent and the wild type.

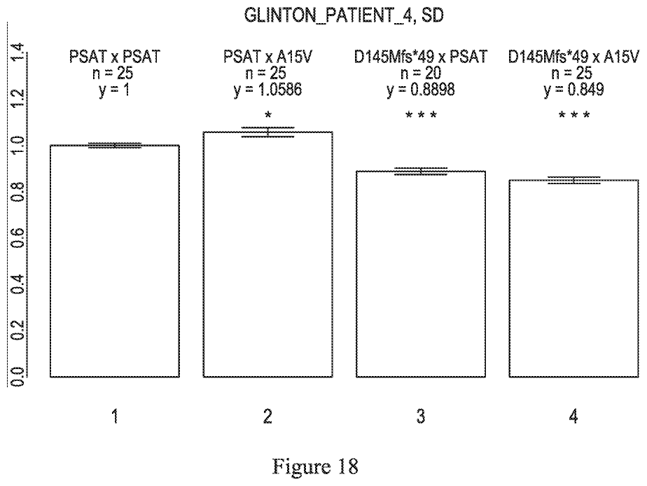

As shown in FIG. 18, a personalized yeast model was also performed for two heterozygous parents that have the alleles for PSAT1×A15V and D145Mfs*49×PSAT100. The progeny (4) has two PSAT alleles (D145Mfs*49×A15V) that lead to a protein with decreased function as compared to the wild type. However, the progeny does have similar protein characteristics with one of the parents (Bar 3), this may possibly show that one mutant is dominant over the other.

In view of the personalized tests, it is envisioned that one would be enabled to predict the types of alleles a child would receive from their parents as well as the outcomes for each case. For example, one would be able to predict if a certain mutation was dominant over another (such as the example shown in FIG. 18), which could possibly lead to a protein that has more function than a child with two recessive alleles (as shown in FIGS. 16 and 17). Thus a model can be created for prospective parents that would let one know the probability of having a child that had both PSAT1 wild type genes as well, two recessive mutant genes, and possible outcomes of the heterozygotes. Thus one would have the ability to develop a therapeutic or prepare an early intervention such as a dietary plan with supplements to prevent neonatal problems or prevent adult onset of a disease.

### Additional Embodiments

### Analysis of Yeast on Solid Agar Plates

In a first aspect, a method of screening a subject for a disease is provide. The method comprises the steps: obtaining genomic DNA from the subject, identifying a gene of interest from the genomic DNA, inserting the gene into a construct, wherein the construct is a linear DNA, providing a test cell, wherein the test cell has a homologous gene of the gene of interest, wherein the homologous gene has been knocked out of the test cell, introducing the construct into the test cell, evaluating the test cell for cell growth in a media, wherein the media lacks an amino acid, wherein the test cell growth is compared to a control cell that has the homologous gene, wherein when test cell growth is comparable to that of the control cell it indicates that the gene of interest encodes a functional protein, and wherein when the test cell growth is slow as compared to the control cell it indicates that the gene of interest encodes a non-functional protein or protein with decreased function and indicates the disease. In some embodiments, the media is solid agar. In some embodiments, the evaluating further comprises quantifying cell growth by photographing yeast growing on solid agar plates and performing image analysis using a custom software.

An analysis may be performed by taking pictures once every 12 hours over the course of 3 days to 5 days, for example. Alternatively, pictures of the agar plates may also be taken every 30 minutes over the course of 3 days. In these cases, at least one time point in that time course (an end point analysis) may be used or several time points may be used to calculate the change in the cell growth over time.

In some embodiments, the media is a liquid media. In some embodiments, the subject is a fetus, neonate, juvenile or adult. In some embodiments, the cell and the control cell are yeast cells. In some embodiments, the amino acid is serine. In some embodiments, the subject is pregnant. In some embodiments, the gene encodes 3-PGDH, PSAT1 or PSPH. In some embodiments, cell growth is measured by optical density. In some embodiments, the evaluating step comprises measuring cell growth for 0.5, 2, 4, 6, 8, 10, 12, 24, 36, 72, 96 or 120 hours or any number of hours in between a range defined by any two aforementioned values. In some embodiments, cell growth of the test cell is comparable to that of the control cell by automated image analysis, wherein the test cell has an optical density that is at least 90% of the growth value of the control cell. In some embodiments, cell growth of the test cell is slow as compared to the control cell, wherein the cell has an optical density that is 79% or less than the optical density of the control cell. In some embodiments, the gene of interest is further analyzed for a single-nucleotide polymorphism. In some embodiments, the single-nucleotide polymorphism is identified as being associated with loss of function or decreased function of a protein encoded by the gene of interest. In some embodiments, a t-test is performed between the test cell and control cell to examine significant growth difference, wherein a t-test p-values <0.0001 indicates a significant growth difference between the test cell and control cell. In some embodiments, the significant growth difference indicates that the gene of interest encodes a non-functional protein or protein with decreased function and indicates the disease. In some embodiments, the disease is NLS, Serine Deficiency Syndrome or retinal neuropathy. In some embodiments, the evaluating further comprises quantifying cell growth by photographing yeast growing on solid agar plates and performing image analysis using a custom software.

### Methods and Generation of a Look Up Table.

Also contemplated are methods to prepare the look up table in order to generate a prenatal prediction for progeny. The data for predicting a disease in a subject that is a fetus, neonatal, juvenile and adult may be obtained by the methods described herein. This data may be used in a look-up table to predict the phenotypic outcome of a fetus based on the personalized yeast model of both parents and may also be used on a human of any age to determine future health issues, such as adult onset metabolic deficiencies, such as retinal neuropathy. The methods here provide the steps of obtaining genomic DNA from one or two subjects in order to analyze the gene of interest in a test for either haploid cells or diploid cells depending on whether one would like to test one or both of the alleles that have the gene of interest. Thus one can predict the function of a single gene or the effects of having a wild type gene and a mutational recessive as well as a double recessive. The results can then be analyzed to generate a table to correlate certain combinations of genes to a disease and this may be used to develop a therapeutic based on the personalized yeast model results.

### Measurement of Cell Growth

In some embodiments, cell growth, may also be referred to as the “growth value”, is measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the afore mentioned parameters. Measurement of the cell growth in a solid media may be measured by photographing yeast growing on solid agar plates and performing image analysis using custom software.

### Screening a Subject for Disease

A method of screening a subject for a disease or being a carrier for a disease is provided. The method comprises the steps: obtaining genomic DNA from the subject, identifying a gene of interest from the genomic DNA, inserting the gene into a construct, wherein the construct is a linear DNA, providing a test cell, wherein the test cell has a homologous gene of the gene of interest, wherein the homologous gene has been knocked out of the test cell, introducing the construct into the test cell, evaluating the test cell for cell growth in a media, wherein the media lacks an amino acid, wherein the test cell growth is compared to a control cell that has the homologous gene, wherein when test cell growth is comparable to that of the control cell it indicates that the gene of interest encodes a functional protein, and wherein when the test cell growth is slow as compared to the control cell it indicates that the gene of interest encodes a non-functional protein or protein with decreased function and indicates the disease. In some embodiments, the subject is a fetus, neonate, juvenile or adult. In some embodiments, the cell and the control cell are yeast cells. In some embodiments, the amino acid is serine. In some embodiments, the subject is pregnant. In some embodiments, the gene encodes 3-PGDH, PSAT1 or PSPH. In some embodiments, cell growth, hereafter the “growth value”, is measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the aforementioned parameters. In some embodiments, the evaluating step comprises measuring cell growth for 0.5, 2, 4, 6, 8, 10, 12, 24, 36, 72, 96 or 120 hours or any number of hours in between a range defined by any two aforementioned values. In some embodiments, growth of the test cell is comparable to that of the control cell by automated image analysis, wherein the test cell has a growth value that is at least 90% of the growth value of the control cell. In some embodiments, cell growth of the test cell is slow as compared to the control cell, wherein the cell has an optical density or growth value that is 79% or less than the growth value of the control cell. The measurement may be performed by analyzing cell growth on a solid media, thus the measurement is performed by analysis of pixels in a “patch” or “spot” of cells growing on solid media, the change in any of these parameters over time. This cell growth can be measured against a control cell that is carrying a wild type PSAT1 gene. The growth is measured to find thresholds for a severe form of a metabolic disease (e.g. NLS). Milder forms of the disease such as adult onset diseases are likely to have a different threshold than those suffering from a severe form of the disease.

In some embodiments, the gene of interest is further analyzed for a single-nucleotide polymorphism. In some embodiments, the single-nucleotide polymorphism is identified as being associated with loss of function or decreased function of a protein encoded by the gene of interest. In some embodiments, a t-test is performed between the test cell and control cell to examine significant growth difference, wherein a t-test p-values <0.0001 indicates a highly significant growth difference between the test cell and control cell. In some embodiments, the significant growth difference indicates that the gene of interest encodes a non-functional protein or protein with decreased function and indicates the disease. In some embodiments, the disease is NLS, Serine Deficiency Syndrome or retinal neuropathy. In some embodiments, the evaluating further comprises quantifying cell growth by photographing yeast growing on solid agar plates and performing image analysis using custom software.

Cell growth, may also be referred to as the “growth value”, and may be measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the aforementioned parameters.

Additional Sequences

PSAT1_codon_optimized integrating plasmid

1-1342=pUC19 vector backbone

1336-1343 and 4759-4764 (bolded)=Sapl and SnaBI restriction sites used to liberate construct for integration

1343-1438 (italics)=sequence 5′ of SER1 start codon to direct integration

1439-2551 (underlined)=PSAT1 open reading frame that has been codon optimized for expression in Saccharomyces cerevisiae.

2552-3014 (bolded italics)=Sequence 3′ of SER1 stop codon that includes SER1 terminator sequence

3015-4520 (lower case)=kanMX drug marker

4521-4761 (italics)=sequence 3′ of SER1 terminator to direct integration

4762-6097 (capital letters)=pUC19 vector backbone

AB481 or pAS19-PSAT1-K sequence (SEQ ID NO: 3)

acccacgctcaccggctccagatttatcagcaataaaccagccagccggaagggccgagcgc
agaagtggtcctgcaactttatccgcctccatccagtctattaattgttgccgggaagctag
agtaagtagttcgccagttaatagtttgcgcaacgttgttgccattgctacaggcatcgtgg
tgtcacgctcgtcgtttggtatggcttcattcagctccggttcccaacgatcaaggcgagtt
acatgatcccccatgttgtgcaaaaaagcggttagctccttcggtcctccgatcgttgtcag
aagtaagttggccgcagtgttatcactcatggttatggcagcactgcataattctcttactg
tcatgccatccgtaagatgcttttctgtgactggtgagtactcaaccaagtcattctgagaa
tagtgtatgcggcgaccgagttgctcttgcccggcgtcaatacgggataataccgcgccaca
tagcagaactttaaaagtgctcatcattggaaaacgttcttcggggcgaaaactctcaagga
tcttaccgctgttgagatccagttcgatgtaacccactcgtgcacccaactgatcttcagca
tcttttactttcaccagcgtttctgggtgagcaaaaacaggaaggcaaaatgccgcaaaaaa
gggaataagggcgacacggaaatgttgaatactcatactcttcctttttcaatattattgaa
gcatttatcagggttattgtctcatgagcggatacatatttgaatgtatttagaaaaataaa
caaataggggttccgcgcacatttccccgaaaagtgccacctgacgtctaagaaaccattat
tatcatgacattaacctataaaaataggcgtatcacgaggccctttcgtctcgcgcgtttcg
gtgatgacggtgaaaacctctgacacatgcagctcccggagacggtcacagcttgtctgtaa
gcggatgccgggagcagacaagcccgtcagggcgcgtcagcgggtgttggcgggtgtcgggg
ctggcttaactatgcggcatcagagcagattgtactgagagtgcaccatatgcggtgtgaaa
taccgcacagatgcgtaaggagaaaataccgcatcaggcgccattcgccattcaggctgcgc
aactgttgggaagggcgatcggtgcgggcctcttcgctattacgccagctggcgaaaggggg
atgtgctgcaaggcgattaagttgggtaacgccagggttttcccagtcacgacgttgtaaaa
cgacggccagtgaattcgagctcggtacccggggctcttc [symbol] ACAAAGACATTAAGGAGCCTT
TGAAATTACTTAATTGAACAACACAGGTCTAATTAGTTGATCAATCATCGATTAACCATTAG
TGATAAGAAACAATG GAC GCG CCA AGA CAG GTT GTG AAT TTC GGA CCT
GGG CCA GCT AAA TTG CCT CAC TCT GTT TTG TTG GAA ATC CAG AAA
GAG TTA TTA GAT TAT AAA GGT GTA GGT ATA TCA GTC TTG GAA ATG
TCT CAC CGT TCG TCT GAT TTC GCC AAA ATA ATT AAT AAC ACC GAG
AAC TTA GTT AGG GAA CTG CTA GCC GTT CCT GAC AAC TAT AAA GTG
ATT TTT TTG CAA GGT GGT GGC TGC GGT CAA TTT TCT GCC GTT CCT
TTA AAT TTG ATA GGT TTA AAA GCG GGT AGA TGT GCC GAC TAT GTC
GTC ACC GGT GCG TGG TCT GCT AAA GCC GCT GAA GAG GCA AAG AAA
TTC GGT ACT ATC AAC ATT GTT CAT CCA AAG TTG GGC TCT TAC ACT
AAA ATT CCG GAT CCA TCC ACA TGG AAT TTA AAT CCC GAT GCA TCA
TAC GTG TAC TAC TGT GCT AAT GAA ACT GTT CAT GGT GTT GAA TTT
GAC TTT ATT CCA GAT GTT AAG GGT GCT GTT TTG GTT TGC GAC ATG
TCC AGC AAC TTC TTG TCT AAG CCA GTT GAC GTT TCT AAA TTC GGT
GTC ATC TTT GCA GGC GCC CAA AAA AAC GTT GGT TCT GCA GGC GTA
ACC GTG GTT ATT GTC AGA GAT GAC TTA CTT GGT TTT GCG TTA AGA
GAA TGC CCT TCT GTT TTA GAG TAT AAG GTG CAG GCC GGT AAC TCA
TCA CTG TAT AAT ACA CCA CCA TGC TTC TCT ATA TAT GTT ATG GGT
CTC GTG TTA GAA TGG ATT AAG AAT AAT GGT GGT GCA GCT GCG ATG
GAG AAA TTG TCT TCT ATT AAG TCT CAG ACG ATA TAT GAG ATA ATA
GAT AAC TCA CAG GGT TTT TAC GTT TGC CCA GTG GAA CCA CAG AAT
AGA TCA AAA ATG AAT ATC CCA TTC CGT ATA GGT AAC GCG AAG GGC
GAT GAC GCC CTT GAA AAG AGG TTT CTA GAT AAG GCA CTT GAG TTA
AAT ATG CTG TCT TTG AAA GGA CAT CGT TCA GTT GGT GGG ATC AGA
GCC TCC TTG TAT AAC GCG GTG ACG ATC GAA GAC GTA CAA AAG TTG
GCC GCT TTT ATG AAG AAA TTT TTG GAG ATG CAC CAG TTA TAA

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cgtacgctgcaggtcgacggatccccgggttaa
ttaaggcgcgccagatctgtttagcttgccttgtccccgccgggtcacccggccagcgacat
ggaggcccagaataccctccttgacagtcttgacgtgcgcagctcaggggcatgatgtgact
gtcgcccgtacatttagcccatacatccccatgtataatcatttgcatccatacattttgat
ggccgcacggcgcgaagcaaaaattacggctcctcgctgcagacctgcgagcagggaaacgc
tcccctcacagacgcgttgaattgtccccacgccgcgcccctgtagagaaatataaaaggtt
aggatttgccactgaggttcttctttcatatacttccttttaaaatcttgctaggatacagt
tctcacatcacatccgaacataaacaaccatgggtaaggaaaagactcacgtttcgaggccg
cgattaaattccaacatggatgctgatttatatgggtataaatgggctcgcgataatgtcgg
gcaatcaggtgcgacaatctatcgattgtatgggaagcccgatgcgccagagttgtttctga
aacatggcaaaggtagcgttgccaatgatgttacagatgagatggtcagactaaactggctg
acggaatttatgcctcttccgaccatcaagcattttatccgtactcctgatgatgcatggtt
actcaccactgcgatccccggcaaaacagcattccaggtattagaagaatatcctgattcag
gtgaaaatattgttgatgcgctggcagtgttcctgcgccggttgcattcgattcctgtttgt
aattgtccttttaacagcgatcgcgtatttcgtctcgctcaggcgcaatcacgaatgaataa
cggtttggttgatgcgagtgattttgatgacgagcgtaatggctggcctgttgaacaagtct
ggaaagaaatgcataagcttttgccattctcaccggattcagtcgtcactcatggtgatttc
tcacttgataaccttatttttgacgaggggaaattaataggttgtattgatgttggacgagt
cggaatcgcagaccgataccaggatcttgccatcctatggaactgcctcggtgagttttctc
cttcattacagaaacggctttttcaaaaatatggtattgataatcctgatatgaataaattg
cagtttcatttgatgctcgatgagtttttctaatcagtactgacaataaaaagattcttgtt
ttcaagaacttgtcatttgtatagtttttttatattgtagttgttctattttaatcaaatgt
tagcgtgatttatattttttttcgcctcgacatcatctgcccagatgcgaagttaagtgcgc
agaaagtaatatcatgcgtcaatcgtatgtgaatgctggtcgctatactgctgtcgattcga
tactaacgccgccatccagtgtcgaaaacgagctcgaattcatcgatAAGAAGTATCTCTTT
TCAAGAGGATTACTTTTCATTGAAAACTTTTGGACGGGCaAAAAAAAAAAAAGCGCCAGCAG
GAAGAAAAAAGGAATCTAGTAAAAAATAAAAATTAGGTTTATAAAGTAGTAAGTGAAGTGCA
AGAGGGAGCGTTATTGGACGATCATGTTGTGATCGGATCCCGTTTATTGGTCTTTGATTCAA
TTTAAAAGAAAAGAAATGTACAGGTTAAACTTCAACA [symbol] gtatgcaggcatgcaagcttgg
cgtaatcatggtcatagctgtttcctgtgtgaaattgttatccgctcacaattccacacaac
atacgagccggaagcataaagtgtaaagcctggggtgcctaatgagtgagctaactcacatt
aattgcgttgcgctcactgcccgctttccagtcgggaaacctgtcgtgccagctgcattaat
gaatcggccaacgcgcggggagaggcggtttgcgtattgggcgctcttccgcttcctcgctc
actgactcgctgcgctcggtcgttcggctgcggcgagcggtatcagctcactcaaaggcggt
aatacggttatccacagaatcaggggataacgcaggaaagaacatgtgagcaaaaggccagc
aaaaggccaggaaccgtaaaaaggccgcgttgctggcgtttttccataggctccgcccccct
gacgagcatcacaaaaatcgacgctcaagtcagaggtggcgaaacccgacaggactataaag
ataccaggcgtttccccctggaagctccctcgtgcgctctcctgttccgaccctgccgctta
ccggatacctgtccgcctttctcccttcgggaagcgtggcgctttctcatagctcacgctgt
aggtatctcagttcggtgtaggtcgttcgctccaagctgggctgtgtgcacgaaccccccgt
tcagcccgaccgctgcgccttatccggtaactatcgtcttgagtccaacccggtaagacacg
acttatcgccactggcagcagccactggtaacaggattagcagagcgaggtatgtaggcggt
gctacagagttcttgaagtggtggcctaactacggctacactagaagaacagtatttggtat
ctgcgctctgctgaagccagttaccttcggaaaaagagttggtagctcttgatccggcaaac
aaaccaccgctggtagcggtggtttttttgtttgcaagcagcagattacgcgcagaaaaaaa
ggatctcaagaagatcctttgatcttttctacggggtctgacgctcagtggaacgaaaactc
acgttaagggattttggtcatgagattatcaaaaaggatcttcacctagatccttttaaatt
aaaaatgaagttttaaatcaatctaaagtatatatgagtaaacttggtctgacagttaccaa
tgcttaatcagtgaggcacctatctcagcgatctgtctatttcgttcatccatagttgcctg
actccccgtcgtgtagataactacgatacgggagggcttaccatctggccccagtgctgcaa
tgataccgcgagATGGACGCGCCAAGACAGGTTGTGAATTTCGGACCTGGGCCAGCTAAATT
GCCTCACTCTGTTTTGTTGGAAATCCAGAAAGAGTTATTAGATTATAAAGGTGTAGGTATAT
CAGTCTTGGAAATGTCTCACCGTTCGTCTGATTTCGCCAAAATAATTAATAACACCGAGAAC
TTAGTTAGGGAACTGCTAGCCGTTCCTGACAACTATAAAGTGATTTTTTTGCAAGGTGGTGG
CTGCGGTCAATTTTCTGCCGTTCCTTTAAATTTGATAGGTTTAAAAGCGGGTAGATGTGCCG
ACTATGTCGTCACCGGTGCGTGGTCTGCTAAAGCCGCTGAAGAGGCAAAGAAATTCGGTACT
ATCAACATTGTTCATCCAAAGTTGGGCTCTTACACTAAAATTCCGGATCCATCCACATGGAA
TTTAAATCCCGATGCATCATACGTGTACTACTGTGCTAATGAAACTGTTCATGGTGTTGAAT
TTGACTTTATTCCAGATGTTAAGGGTGCTGTTTTGGTTTGCGACATGTCCAGCAACTTCTTG
TCTAAGCCAGTTGACGTTTCTAAATTCGGTGTCATCTTTGCAGGCGCCCAAAAAAACGTTGG
TTCTGCAGGCGTAACCGTGGTTATTGTCAGAGATGACTTACTTGGTTTTGCGTTAAGAGAAT
GCCCTTCTGTTTTAGAGTATAAGGTGCAGGCCGGTAACTCATCACTGTATAATACACCACCA
TGCTTCTCTATATATGTTATGGGTCTCGTGTTAGAATGGATTAAGAATAATGGTGGTGCAGC
TGCGATGGAGAAATTGTCTTCTATTAAGTCTCAGACGATATATGAGATAATAGATAACTCAC
AGGGTTTTTACGTTTGCCCAGTGGAACCACAGAATAGATCAAAAATGAATATCCCATTCCGT
ATAGGTAACGCGAAGGGCGATGACGCCCTTGAAAAGAGGTTTCTAGATAAGGCACTTGAGTT
AAATATGCTGTCTTTGAAAGGACATCGTTCAGTTGGTGGGATCAGAGCCTCCTTGTATAACG
CGGTGACGATCGAAGACGTACAAAAGTTGGCCGCTTTTATGAAGAAATTTTTGGAGATGCAC
CAGTTATAA

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through eleventh aspects is applicable to all aspects and embodiments identified herein. Moreover, any of the features of an embodiment of the first through eleventh aspects is independently combinable, partly or wholly with other embodiments described herein in any way, e.g., one, two, or three or more embodiments may be combinable in whole or in part. Further, any of the features of an embodiment of the first through eleventh aspects may be made optional to other aspects or embodiments.

What is claimed is:

1. A method of screening a subject for a disease comprising:
a) obtaining genomic DNA from the subject;
b) identifying a gene of interest from the genomic DNA;
c) inserting the gene into a construct, wherein the construct is a linear DNA;
d) providing a test cell, wherein the test cell has a homologous gene of the gene of interest, wherein the homologous gene has been knocked out of the test cell;
e) introducing the construct into the test cell;
f) evaluating the test cell for cell growth in a media, wherein the media lacks an amino acid, wherein the test cell growth is compared to a control cell that has the homologous gene, wherein when test cell growth is comparable to that of the control cell it indicates that the gene of interest encodes a functional protein, and wherein when the test cell growth is slow as compared to the control cell it indicates that the gene of interest encodes a non-functional protein or protein with decreased function and indicates the disease.

2. The method of claim 1, wherein the subject is a fetus, neonate, juvenile or adult.

3. The method of claim 1 or 2, wherein the cell and the control cell are yeast cells.

4. The method of any one of claims 1-3, wherein the amino acid is serine.

5. The method of any one of claim 1-4, wherein the subject is pregnant.

6. The method of any one of claims 1-5, wherein the gene encodes 3-PGDH, PSAT1 or PSPH.

7. The method of any one of claims 1-6, wherein cell growth is measured by optical density of a liquid culture, a number of pixels of a colony of cells growing on solid media.

8. The method of any one of claims 1-7, wherein the evaluating step comprises measuring cell growth for 0.5, 2, 4, 6, 8, 10, 12, 24, 36, 72, 96 or 120 hours or any number of hours in between a range defined by any two aforementioned values.

9. The method of any one of claims 1-8, wherein cell growth of the test cell is comparable to that of the control cell by automated image analysis, wherein the test cell has an optical density that is at least 90% of the growth value of the control cell.

10. The method of any one of claims 1-8, wherein cell growth of the test cell is slow as compared to the control cell, wherein the cell has an optical density that is 79% or less than the optical density of the control cell.

11. The method of any one of claims 1-10, wherein the gene of interest is further analyzed for a single-nucleotide polymorphism.

12. The method of any one of claim 11, wherein the single-nucleotide polymorphism is identified as being associated with loss of function or decreased function of a protein encoded by the gene of interest.

13. The method of any one of claims 1-12, wherein a t-test is performed between the test cell and control cell to examine significant growth difference, wherein a t-test p-values <0.0001 indicates a significant growth difference between the test cell and control cell.

14. The method of claim 13, wherein the significant growth difference indicates that the gene of interest encodes a non-functional protein or protein with decreased function and indicates the disease.

15. The method of any one of claims 1-14, wherein the disease is NLS, Serine Deficiency Syndrome or retinal neuropathy.

16. A method of determining amino acid deficiency in a subject, the method comprising:
a) isolating genomic DNA from a subject;
b) detecting a gene from the genomic DNA, wherein the gene encodes an enzyme of an amino acid synthesis pathway;
c) inserting the gene into a construct;
d) introducing the construct into a test cell;
e) growing up the cell in a media absent of an amino acid; and
f) analyzing growth of the test cell in a culture, wherein the growth of the test cell is compared to a control cell, wherein the control cell is not deficient in amino acid synthesis wherein the test cell growth is compared to the control cell, wherein cell growth of the test cell is comparable to that of the control cell indicates that the gene encodes a functional enzyme and wherein cell growth of the test cell is slow as compared to the control cell indicates that the gene encodes a non-functional protein or protein with decreased function and indicates The method of claim 1, wherein the subject is a fetus, neonate, juvenile or adult.

17. The method of claim 16, wherein the cell and the control cell are yeast cells.

18. The method of any one of claims 16-17, wherein the amino acid is serine.

19. The method of any one of claim 16-18, wherein the subject is pregnant.

20. The method of any one of claims 16-19, wherein the gene encodes 3-PGDH, PSAT1 or PSPH.

21. The method of any one of claims 16-20, wherein cell growth is measured by optical density of a liquid culture, a number of pixels of a colony of cells growing on solid media.

22. The method of any one of claims 16-21, wherein the analyzing step comprises measuring cell growth for 0.5, 2, 4, 6, 8, 10, 12, 24, 36, 72, 96 or 120 hours or any number of hours in between a range defined by any two aforementioned values.

23. The method of any one of claims 16-22, wherein cell growth of the test cell is comparable to that of the control cell by automated image analysis, wherein the test cell has an optical density that is at least 90% of the growth value of the control cell.

24. The method of any one of claims 16-23, wherein cell growth of the test cell is slow as compared to the control cell, wherein the cell has an optical density that is 79% or less than the optical density of the control cell.

25. The method of any one of claims 16-24, wherein the gene of interest is further analyzed for a single-nucleotide polymorphism.

26. The method of any one of claim 25, wherein the single-nucleotide polymorphism is identified as being associated with loss of function or decreased function of a protein encoded by the gene of interest.

27. The method of any one of claims 16-26, wherein a t-test is performed between the test cell and control cell to examine significant growth difference, wherein a t-test p-values <0.0001 indicates a significant growth difference between the test cell and control cell.

28. The method of claim 27, wherein the significant growth difference indicates that the gene of interest encodes a non-functional protein or protein with decreased function and indicates the disease.

29. The method of any one of claims 16-28, wherein the disease is NLS, Serine Deficiency Syndrome, retinal neuropathy, or an amino acid deficiency in the subject.

30. The method of any one of claims 16-29, wherein the analyzing step further comprises identifying at least one mutation in the gene.

31. A method of determining a carrier of an amino acid deficiency disorder, the method comprising:
a) isolating genomic DNA from a subject;
b) detecting a gene from the genomic DNA, wherein the gene encodes an enzyme of an amino acid synthesis pathway, wherein the subject has two different alleles of the gene;
c) inserting the gene into a construct, wherein the construct is a linear DNA;
d) introducing the construct into a test cell;
e) growing up the test cell in a media absent of an amino acid; and
f) evaluating the test cell for cell growth in a media, wherein the media lacks an amino acid, wherein test cell growth is compared to a control cell that has the homologous gene, wherein test cell growth of the cell is comparable to that of the control cell indicates that the gene of interest encodes a functional protein and wherein cell growth of the test cell is slow as compared to the control cell indicates that the gene of interest encodes a non-functional protein or protein with decreased function and indicates that the subject is a carrier of an amino acid deficiency disorder.

32. The method of claim 31, wherein the amino acid deficiency disorder is NLS.

33. A method of treating a subject with an amino acid deficiency, the method comprising:
a) determining a subject or carrier of an amino acid deficiency disorder, wherein the determining comprises:
detecting a gene from the genomic DNA, wherein the gene encodes an enzyme of an amino acid synthesis pathway;
inserting the gene into a construct, wherein the construct is a linear DNA;
introducing the construct into a test cell;
growing up the test cell in a media absent of an amino acid; and
evaluating the test cell for cell growth in a media, wherein the media lacks an amino acid, wherein test cell growth is compared to a control cell that has the homologous gene, wherein cell growth of the test cell is slow as compared to the control cell indicates that the gene of interest encodes a non-functional protein or protein with decreased function and indicates that the subject is a carrier of an amino acid deficiency disorder;
b) providing an adequate amount of an amino acid supplement to the subject.

34. The method of claim 33, wherein the amino acid supplement is serine.

35. The method of claim 33 or 34, wherein the enzyme is wherein the gene encodes 3-PGDH, PSAT1 or PSPH.

36. The method of any one of claims 33-35, wherein the subject is a fetus and wherein the mother of the fetus is provided an adequate amount of amino acid supplement.

37. The method of any one of claims 33-36, wherein the evaluating further comprises comparing growth of the test cell to a second control cell, wherein the second control cell is deficient in serine biosynthesis.

38. The method of any one of claims 33-37, wherein the amino acid deficiency disorder is NLS.

39. A method of prenatal prediction of an amino acid deficiency, the method comprising
a) obtaining genomic DNA from a female and male subject;
b) identifying a same gene of interest from the genomic DNA of the female and male subject, wherein the subjects are homozygous or heterozygous for the gene;
c) inserting a first gene variant of interest from the female into a first construct, wherein the first construct is a linear DNA;
d) inserting a second gene variant of interest from the male into a second construct, wherein the second construct is a linear DNA;
e) providing a first cell, wherein the first cell has a homologous gene of the gene of interest, wherein the homologous gene has been knocked out of the cell;
f) providing a second cell, wherein the second cell has a homologous gene of the gene of interest, wherein the homologous gene has been knocked out of the cell;
g) introducing the first construct into the first cell, wherein the first construct is a linear DNA;
h) introducing the second construct into the second cell, wherein the second construct is a linear DNA; and
i) evaluating the first and second cell for cell growth in a media, wherein the media lacks an amino acid, wherein the cell growth of the first and second cell are compared to a control cell that has the homologous gene, wherein cell growth of the first and/or second cell is comparable to that of the control cell indicates that the first and second gene of interest encodes a functional protein and wherein cell growth of the first and second cell is slow as compared to the control cell indicates that the first and second gene of interest encodes a non-functional protein or protein with decreased function and indicates that the male and/or female is a carrier of a disease for an amino acid deficiency.

40. The method of claim 39, wherein the method further comprises making a diploid strain of a third cell, wherein the third cell comprises the first and second gene of interest and evaluating the third cell for cell growth in a media, wherein the media lacks an amino acid, wherein the cell growth of the third cell are compared to a control cell that has the homologous gene, wherein cell growth of the third cell is comparable to that of the control cell indicates that the first and second gene of interest encodes a functional protein and wherein cell growth of the third cell is slow as compared to the control cell indicates that the first and second gene of interest together indicates a predicted fetus with an amino acid deficiency.

41. The method of any one of claims 39-40, wherein data from the first, second and third cell is stored in a look-up table, wherein the look-up table is generated for disease prediction.

42. The method of any one of claims 39-41, wherein the disease for an amino acid deficiency is NLS.

43. A method of prenatal prediction of an amino acid deficiency wherein at least one parent has a gene mutation, the method comprising:
a) obtaining genomic DNA from a female and male subject;
b) identifying a same gene of interest from the genomic DNA of the female and male subject, wherein the subjects are homozygous or heterozygous for the gene;
c) inserting a first gene of interest from the female into a first construct;
d) inserting a second gene of interest from the male into a second construct;
e) providing a first cell, wherein the first cell has a homologous gene of the gene of interest, wherein the homologous gene has been knocked out of the cell;
f) providing a second cell, wherein the second cell has a homologous gene of the gene of interest, wherein the homologous gene has been knocked out of the cell;
g) making a diploid strain of a third cell, wherein the third cell comprises the first and second gene of interest
h) introducing the first construct into the first cell, wherein the first construct is a linear DNA;
i) introducing the second construct into the second cell, wherein the second construct is a linear DNA; and
j) evaluating the first, second, and third cell for cell growth in a media, wherein the media lacks an amino acid, wherein the cell growth of the first and second cell are compared to a control cell that has the homologous gene, wherein cell growth of the first, second and third cell is comparable to that of the control cell indicates that the first and second gene of interest encodes a functional protein and wherein cell growth of the first, second and third cell is slow as compared to the control cell indicates that the first and second gene of interest encodes a non-functional protein or protein with decreased function and predicts an amino acid deficiency for progeny.

44. The method of claim 43, wherein the first gene of interest or the second gene of interest comprises the gene mutation.

45. The method of claim 43 or 44, wherein the gene of interest encodes PSAT.

46. The method of any one of claims 43-45, wherein the gene of interest encodes PSAT1 with at least one of the amino mutations A99V, S179L, T156M, G78A, R213C or R222.

47. The method of any one of claims 43-46, wherein data from the first, second and third cell is stored in a look-up table, wherein the look-up table is generated for disease prediction.

48. The method of any one of claims 43-47, wherein the amino acid deficiency is caused by NLS.

49. A kit for determining amino acid deficiency comprising:
a look-up table, wherein the look up table indicates genes or combinations of genes that may be indicative of an amino acid deficiency or disease;
one or more nucleic acid probes for detecting one or more mutation in PSAT, wherein the one or more mutation includes at least: G78A, R213C, T156M or 8222.

50. The kit of claim 49, further comprising a serine supplement in an amount sufficient to treat a subject suffering from serine deficiency.

51. A method of determining if a subject is suffering from an amino acid deficiency, the method comprising:
providing a look-up table, wherein the look-up table comprises genes or a combination of genes that are indicative of a disease;
isolating genes of interest from a subject;
determining if the genes include one of more mutations in the look-up table; and
determining a probability of disease in the subject.

52. The method of claim 51, wherein the look up table comprises combination of genes that include genes of PSAT, wherein the PSAT genes encode PSAT comprising mutations G78A, R213C, T156M or R222.

53. The method of claim 52, wherein the look up table comprises a list of mutations that have been determined by the method of claim 43, said method further comprising administering an amino acid supplement to the subject to treat the amino acid deficiency if the subject has more than a 50% probability of having the amino acid deficiency.

54. A method of treating a subject with an amino acid deficiency, the method comprising:
a) determining if a subject has at least one mutation in PSAT located at G78, R213, T156 or 8222; and
b) providing an adequate amount of a serine supplement to the subject if the subject has the at least one mutation.

55. The method of claim 54, wherein the at least one mutation is G78A, R213C, T156M or R222, and wherein the subject is an unborn child of a mother, wherein the mother is tested for the presence of the at least one mutation.

56. A method of identifying a point mutation as a cause or marker of an amino acid deficiency, the method comprising:
a) obtaining genomic DNA from a subject having the amino acid deficiency;
b) identifying a point mutation in the genomic DNA in at least one of 3-PGDH, PSAT1 or PSPH;
c) providing a test yeast cell, wherein a homologous gene of at least one of 3-PGDH, PSAT1 or PSPH has been knocked out of the test yeast cell;
d) introducing the gene with the point mutation into the test yeast cell; and
e) evaluating the test yeast cell for cell growth in a media, wherein the media lacks an amino acid, wherein the test yeast cell growth is compared to a control cell that has the homologous gene, wherein when test yeast cell growth is comparable to that of the control cell it indicates that the point mutation still allows for a functional protein, and wherein when the test yeast cell growth is slow as compared to the control cell it indicates that the point mutation results in a non-functional protein or protein with decreased function, thereby identifying the point mutation as a cause or marker of an amino acid deficiency.

57. A method of preparing a personalized yeast model for determining a subject at risk of a disease, the method comprising
a) obtaining genomic DNA from the subject;
b) identifying a gene of interest in at least two alleles from the genomic DNA;
c) inserting the gene into a construct, wherein the construct is a linear DNA;
d) providing a test cell, wherein the test cell has a homologous gene of the gene of interest, wherein the homologous gene has been knocked out of the test cell;
e) introducing the construct into the test cell;
f) evaluating the test cell for cell growth in a media, wherein the media lacks an amino acid, wherein the test cell growth is compared to a control cell that has the homologous gene, wherein when test cell growth is comparable to that of the control cell it indicates that the gene of interest encodes a functional protein, and wherein when the test cell growth is slow as compared to the control cell it indicates that the gene of interest encodes a non-functional protein or protein with decreased function and indicates the disease;
g) generating data for a look-up table; and
h) generating one or more personalized disease amelioration recommendations for the subject; and presenting the one or more personalized disease prevention recommendations for the subject in the personalized disease prevention plan for the subject for disease management.

58. The method of claim 57, wherein the method further comprising: a) obtaining a second genomic DNA from a second subject, wherein the second subject has a second set of two alleles that are related to the two alleles of the subject, and second set of two alleles have a second gene of interest, wherein the second gene of interest is placed in a second construct b) introducing the second construct into a second test cell, evaluating the second test cell for cell growth in a media, wherein the media lacks an amino acid, wherein the test cell growth is compared to a control cell that has the homologous gene, wherein when second test cell growth is comparable to that of the control cell it indicates that the gene of interest encodes a functional protein and wherein when the test cell growth is slow as compared to the control cell it indicates that the gene of interest encodes a non-functional protein or protein with decreased function.

59. The method of claim 58, further comprising obtaining a second genomic DNA from a second subject and identifying a second gene(s) of interest in at least two alleles from the genomic DNA, inserting at least one of the second gene(s) into a second construct, introducing the second construct into a second test cell, and mating the second cell with the first cell to produce a progeny cell.

60. The method of claim 59, further comprising evaluating the progeny cell for cell growth in a media, wherein the media lacks an amino acid, wherein the test cell growth is compared to a control cell that has the homologous gene, wherein when progeny cell growth is comparable to that of the control cell it indicates that the gene of interest encodes a functional protein, and wherein when the progeny cell growth is slow as compared to the control cell it indicates that the gene of interest encodes a non-functional protein or protein with decreased function and indicates the disease.

61. The method of any of claims 1-15, wherein the evaluating further comprises quantifying cell growth by photographing yeast growing on solid agar plates and performing image analysis using a custom software.


SEQUENCE LISTING (3 sequences)

SEQ ID NO: 1 - DNA, 1146 bp, Artificial Sequence
  codon optimized PSAT1
atggacgcgccaagacaggttgtgaatttcggacctgggccagctaaattgcctcactctgttttgttggaaatccagaaagagttattagattataaaggtgtaggtatatcagtcttggaaatgtctcaccgttcgtctgatttcgccaaaataattaataacaccgagaacttagttagggaactgctagccgttcctgacaactataaagtgatttttttgcaaggtggtggctgcggtcaattttctgccgttcctttaaatttgataggtttaaaagcgggtagatgtgccgactatgtcgtcaccggtgcgtggtctgctaaagccgctgaagaggcaaagaaattcggtactatcaacattgttcatccaaagttgggctcttacactaaaattccggatccatccacatggaatttaaatcccgatgcatcatacgtgtactactgtgctaatgaaactgttcatggtgttgaatttgactttattccagatgttaagggtgctgttttggtttgcgacatgtccagcaacttcttgtctaagccagttgacgtttctaaattcggtgtcatctttgcaggcgcccaaaaaaacgttggttctgcaggcgtaaccgtggttattgtcagagatgacttacttggttttgcgttaagagaatgcccttctgttttagagtataaggtgcaggccggtaactcatcactgtataatacaccaccatgcttctctatatatgttatgggtctcgtgttagaatggattaagaataatggtggtgcagctgcgatggagaaattgtcttctattaagtctcagacgatatatgagataatagataactcacagggtttttacgtttgcccagtggaaccacagaatagatcaaaaatgaatatcccattccgtataggtaacgcgaagggcgatgacgcccttgaaaagaggtttctagataaggcacttgagttaaatatgctgtctttgaaaggacatcgttcagttggtgggatcagagcctccttgtataacgcggtgacgatcgaagacgtacaaaagttggccgcttttatgaagaaatttttggagatgcaccagttataaatgaagaaatttttggagatgcaccagttataa

SEQ ID NO: 2 - DNA, 1146 bp, Homo sapiens
atggacgcccccaggcaggtggtcaactttgggcctggtcccgccaagctgccgcactcagtgttgttagagatacaaaaggaattattagactacaaaggagttggcattagtgttcttgaaatgagtcacaggtcatcagattttgccaagattattaacaatacagagaatcttgtgcgggaattgctagctgttccagacaactataaggtgatttttctgcaaggaggtgggtgcggccagttcagtgctgtccccttaaacctcattggcttgaaagcaggaaggtgtgcggactatgtggtgacaggagcttggtcagctaaggccgcagaagaagccaagaagtttgggactataaatatcgttcaccctaaacttgggagttatacaaaaattccagatccaagcacctggaacctcaacccagatgcctcctacgtgtattattgcgcaaatgagacggtgcatggtgtggagtttgactttatacccgatgtcaagggagcagtactggtttgtgacatgtcctcaaacttcctgtccaagccagtggatgtttccaagtttggtgtgatttttgctggtgcccagaagaatgttggctctgctggggtcaccgtggtgattgtccgtgatgacctgctggggtttgccctccgagagtgcccctcggtcctggaatacaaggtgcaggctggaaacagctccttgtacaacacgcctccatgtttcagcatctacgtcatgggcttggttctggagtggattaaaaacaatggaggtgccgcggccatggagaagcttagctccatcaaatctcaaacaatttatgagattattgataattctcaaggattctacgtttgtccagtggagccccaaaatagaagcaagatgaatattccattccgcattggcaatgccaaaggagatgatgctttagaaaaaagatttcttgataaagctcttgaactcaatatgttgtccttgaaagggcataggtctgtgggaggcatccgggcctctctgtataatgctgtcacaattgaagacgttcagaagctggccgccttcatgaaaaaatttttggagatgcatcagctatgaatgaaaaaatttttggagatgcatcagctatga

SEQ ID NO: 3 - DNA, 7210 bp, Artificial Sequence
  plasmid
acccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggctcttcgacaaagacattaaggagcctttgaaattacttaattgaacaacacaggtctaattagttgatcaatcatcgattaaccattagtgataagaaacaatggacgcgccaagacaggttgtgaatttcggacctgggccagctaaattgcctcactctgttttgttggaaatccagaaagagttattagattataaaggtgtaggtatatcagtcttggaaatgtctcaccgttcgtctgatttcgccaaaataattaataacaccgagaacttagttagggaactgctagccgttcctgacaactataaagtgatttttttgcaaggtggtggctgcggtcaattttctgccgttcctttaaatttgataggtttaaaagcgggtagatgtgccgactatgtcgtcaccggtgcgtggtctgctaaagccgctgaagaggcaaagaaattcggtactatcaacattgttcatccaaagttgggctcttacactaaaattccggatccatccacatggaatttaaatcccgatgcatcatacgtgtactactgtgctaatgaaactgttcatggtgttgaatttgactttattccagatgttaagggtgctgttttggtttgcgacatgtccagcaacttcttgtctaagccagttgacgtttctaaattcggtgtcatctttgcaggcgcccaaaaaaacgttggttctgcaggcgtaaccgtggttattgtcagagatgacttacttggttttgcgttaagagaatgcccttctgttttagagtataaggtgcaggccggtaactcatcactgtataatacaccaccatgcttctctatatatgttatgggtctcgtgttagaatggattaagaataatggtggtgcagctgcgatggagaaattgtcttctattaagtctcagacgatatatgagataatagataactcacagggtttttacgtttgcccagtggaaccacagaatagatcaaaaatgaatatcccattccgtataggtaacgcgaagggcgatgacgcccttgaaaagaggtttctagataaggcacttgagttaaatatgctgtctttgaaaggacatcgttcagttggtgggatcagagcctccttgtataacgcggtgacgatcgaagacgtacaaaagttggccgcttttatgaagaaatttttggagatgcaccagttataatatcagcggaatatgcctgtaatttggttcaaagaaaaactttttcaagtcatatgtaagttttaaataaatcagtttacgaacatgcacaagaataacttctcttctttttcttgaaataaaggccgaattatacgatatgatatgagacgttctaattacaatacgataccgtgagataaaaatagcgatatcctcgtactcaaagaagtctaattgttttagcccgttgatcaaaaaaataaaatgtatttttatttttttgtcatccagctcaagatcgtaatattggcaattaacaaccgagcgaggcacagggcaaagagaatgtgggtgagactgaactatctctcccaatatattatacttttttttgtcgcgtctaccaattaaatgaaaacataattgggaatactttcctcttcttatattttatcttaactcgtttccgatcttctgatatcgtacgctgcaggtcgacggatccccgggttaattaaggcgcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgataagaagtatctcttttcaagaggattacttttcattgaaaacttttggacgggcaaaaaaaaaaaaagcgccagcaggaagaaaaaaggaatctagtaaaaaataaaaattaggtttataaagtagtaagtgaagtgcaagagggagcgttattggacgatcatgttgtgatcggatcccgtttattggtctttgattcaatttaaaagaaaagaaatgtacaggttaaacttcaacatacgtatgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatggacgcgccaagacaggttgtgaatttcggacctgggccagctaaattgcctcactctgttttgttggaaatccagaaagagttattagattataaaggtgtaggtatatcagtcttggaaatgtctcaccgttcgtctgatttcgccaaaataattaataacaccgagaacttagttagggaactgctagccgttcctgacaactataaagtgatttttttgcaaggtggtggctgcggtcaattttctgccgttcctttaaatttgataggtttaaaagcgggtagatgtgccgactatgtcgtcaccggtgcgtggtctgctaaagccgctgaagaggcaaagaaattcggtactatcaacattgttcatccaaagttgggctcttacactaaaattccggatccatccacatggaatttaaatcccgatgcatcatacgtgtactactgtgctaatgaaactgttcatggtgttgaatttgactttattccagatgttaagggtgctgttttggtttgcgacatgtccagcaacttcttgtctaagccagttgacgtttctaaattcggtgtcatctttgcaggcgcccaaaaaaacgttggttctgcaggcgtaaccgtggttattgtcagagatgacttacttggttttgcgttaagagaatgcccttctgttttagagtataaggtgcaggccggtaactcatcactgtataatacaccaccatgcttctctatatatgttatgggtctcgtgttagaatggattaagaataatggtggtgcagctgcgatggagaaattgtcttctattaagtctcagacgatatatgagataatagataactcacagggtttttacgtttgcccagtggaaccacagaatagatcaaaaatgaatatcccattccgtataggtaacgcgaagggcgatgacgcccttgaaaagaggtttctagataaggcacttgagttaaatatgctgtctttgaaaggacatcgttcagttggtgggatcagagcctccttgtataacgcggtgacgatcgaagacgtacaaaagttggccgcttttatgaagaaatttttggagatgcaccagttataa